(19) United States
(12) Patent Application Publication
(10) Pub. No.: US 2025/0354970 A1
(43) Pub. Date: Nov. 20, 2025

(54) SYSTEMS, METHODS, AND APPARATUSES FOR CHARACTERIZING RELEASE PROFILES FOR LONG-ACTING INJECTABLE MEDICATIONS

(71) Applicant: Gilead Sciences, Inc., Foster City, CA (US)

(72) Inventors: Krutika Harish Jain, Berkeley, CA (US); Christopher J. Foti, Fremont, CA (US); Robert C.H. Gresham, San Francisco, CA (US); Jeffrey N. Hemenway, San Mateo, CA (US); Tien D. Ho, Union City, CA (US); Thoeun Khuth, Gilroy, CA (US); Maria Victoria Olivares Hagopian, Foster City, CA (US); Pallavi Pralhad Pawar, Fremont, CA (US); Yujie Qin, Newark, CA (US); Charles W. Rowe, San Bruno, CA (US); Ethan L. Stroh, Belmont, CA (US); Bo Wan, Redwood City, CA (US)

(21) Appl. No.: 19/209,561

(22) Filed: May 15, 2025

Related U.S. Application Data
(60) Provisional application No. 63/649,192, filed on May 17, 2024.

(51) Int. Cl. G01N 33/15

(52) U.S. Cl. CPC G01N 33/15

(57) ABSTRACT

The present disclosure described technology for developing long-acting injectable drug formulations. More specifically, the technology includes systems and methods for predicting a drug release profile in a patient. Importantly, the technologies provided herein enable substantially shortened development timelines for long-acting injectable drug formulations.

### CROSS-REFERENCE TO RELATED APPLICATIONS

This application claims priority to U.S. Provisional Application No. 63/649,192, filed on May 17, 2024 the entirety of which is incorporated herein by reference.

### FIELD

The present disclosure relates to the field of long-acting injectable drug formulation testing technology. More specifically, the present disclosure includes systems, methods, and apparatuses for generating a predicted drug release profile as if the injection had occurred in living tissue.

### BACKGROUND

There has been a growing need for improved long-acting injectable drug formulations and specifically for injectable medications targeting the intramuscular spaces and subcutaneous tissues of patients.

The field of drug development has progressed substantially in recent years, but unfortunately the field of drug delivery lags, specifically, drug delivery via long-acting injectable formulation. Optimal long-acting injectable drug formulations vary based on the drug compound being administered. There are also a multitude of formulation ingredients and concentration options to consider making it virtually impossible to test every long-acting injectable drug formulation.

One of the major hurdles plaguing the industry involves the incredibly long development timelines for new formulations. Evaluation periods required to predict drug release profiles showing release variations of drugs typically require at least the same amount of time as the target drug release duration for the patient. For example, if an injected drug is expected to be released from the injection site of a patient over six months, then at least a six-month evaluation period is typically required to generate predictions involving the release profile of the injected drug using the existing prior art. The industry is moving toward even longer timelines, including, one year or more.

Additionally, the current long-acting injectable drug formulation testing methods often involve use of living organisms which can present both safety and efficiency issues. Existing technology for evaluating long-acting drug formulations can be time consuming, low-throughput, and costly which stifles technical advancement in an area that could benefit patients in need.

What is needed is technology allowing for substantially shortened evaluation periods of long-acting injectable formulations at low cost and with a high degree of predictive accuracy over long timelines. The systems and methods described herein provide for substantially shorter evaluations periods for long-acting injectable drug formulations which until now has remained an unmet need in the field. Additionally, the systems and methods are lower cost than existing solutions, increase patient safety, and offer a high degree of predictive accuracy.

### SUMMARY

In various aspects, a system for predicting a drug release profile of a long-acting injectable formulation is disclosed herein. In various embodiments, the system may comprise a chamber for receiving a fluid. In various embodiments, the system may comprise a sample receiver disposed within the chamber. In various embodiments, the system may comprise a mixing system for mixing the fluid within the chamber.

In various embodiments, the sample receiver may comprise a structural support for supporting a scaffold. In various embodiments, the sample receiver may comprise a sample receiver site associated with the scaffold.

In various embodiments, the sample receiving site may be located within an interior of the scaffold. In various embodiments, the sample receiving site comprises a surface of the scaffold. In various embodiments, the sample receiving site comprises a depression associated with the scaffold.

In various embodiments, the depression comprises a sidewall extending into a surface to a floor. In various embodiments, the sidewall and floor form the depression. In various embodiments, the depression may be cylindrical. In various embodiments, the depression may be conical. In various embodiments, the depression comprises a depth ranging between 2 mm and about 4 mm. In various embodiments, the depression comprises a depth of 3 mm. In various embodiments, the depression comprises a diameter ranging between 2 mm and 3 mm. In various embodiments, the depression comprises a diameter of 2.5 mm.

In various embodiments, the scaffold comprises a hydrogel. In various embodiments, the hydrogel comprises between about 0.5% to 2% agarose. In various embodiments, the hydrogel comprises collagen. In various embodiments, the hydrogel comprises polyacrylamide. In various embodiments, the hydrogel comprises a phosphate buffered saline at pH 7.4. In various embodiments, the scaffold comprises albumin. In various embodiments, the scaffold comprises at least one enzyme.

In various embodiments, the at least one enzyme includes an esterase, a phosphatase, an amidase, or a hydrolase.

In various embodiments, the chamber comprises a sample port. In various embodiments, the sample port may be fluidically connected to a fluid collection apparatus.

In various embodiments, the system may comprise a lid for sealing the chamber at an opening. In various embodiments, the system may comprise a probe extending through the lid. In various embodiments, the probe may be at least partially disposed within the chamber. In various embodiments, the probe comprises a fiber optic ultraviolet (UV) probe.

In various embodiments, the chamber may further comprise an inlet for transferring the fluid into the chamber. In various embodiments, the chamber may further comprise an outlet for transferring the fluid out of the chamber. In various embodiments, the system may comprise a bead situated adjacent to the outlet.

In various embodiments, the sample receiver may be positioned between the inlet and the outlet. In various embodiments, a space within the chamber and between the sample receiver and the outlet may be at least partially filled with a solid matrix to support the sample receiver.

In various embodiments, the system may comprise a fluid sample collection apparatus fluidically connected to the outlet. In various embodiments, the fluid sample collection apparatus may be detachable from a fluidic connection for off-line analysis of the fluid. In various embodiments, the system may further comprise a probe positioned at or near a fluidic connection. In various embodiments, the fluidic connection comprises a tube.

In various embodiments, the probe measures a quality of the fluid. In various embodiments, the measurement includes opacity. In various embodiments, the measurement includes pH. In various embodiments, the measurement includes fluorescence. In various embodiments, the measurement includes ultraviolet light absorption.

In various embodiments, the mixing system comprises a mixing element disposed within the chamber. In various embodiments, comprises a paddle. In various embodiments, comprises a stir bar.

In various embodiments, the structural support includes a floor having at least one upstanding sidewall for supporting the scaffold.

In various embodiments, the structural support comprises a container, a receptacle, a vessel, a canister, a drum, a cassette, a well, a cup, a box, or a receptible having at least one open end.

In various embodiments, the fluid includes a biorelevant media. In various embodiments, the biorelevant media comprises between 2 to 4 percent bovine serum albumin (BSA) or human serum albumin (HSA) as biorelevant solubilizer. In various embodiments, the fluid further includes 0.02% sodium azide to prevent microbial growth in the media during testing.

In various aspects a method for predicting a drug release profile of a long-acting injectable formulation is disclosed herein. In various embodiments, the method comprises generating a depot by combining an injection formulation with a scaffold. In various embodiments, the method comprises contacting the depot with a fluid. In various embodiments, a compound diffuses from the depot to the fluid. In various embodiments, the method comprises analyzing the fluid at a plurality of timepoints.

In various embodiments, the step of generating the depot comprises initiating a fluid solidification process, injecting the drug formulation into the scaffold after the solidification process, and completing the fluid solidification process.

In various embodiments, the method comprises recording at least two measurements of a characteristic of the fluid at different times. In various embodiments, the method comprises generating an in vitro drug release profile using the at least two measurements. In various embodiments, the method comprises predicting an in vivo drug release profile for the injection formulation by comparing the in vitro drug release profile to a reference.

In various embodiments, the characteristic may be influenced by a quantity of the compound in the fluid. In various embodiments, the compound comprises an analyte that may be detectable by a probe.

In various embodiments, the step of generating the in vitro release profile requires less than 30 days and the predicted drug release profile extends to at least 180 days. In various embodiments, the step of generating the in vitro release profile requires five days or less and the predicted drug release profile extends to at least 50 days. In various embodiments, the step of generating the in vitro release profile requires five days or less and the predicted drug release profile extends to at least 100 days. In various embodiments, the step of generating the in vitro release profile requires five days or less and the predicted drug release profile extends to at least 150 days. In various embodiments, the step of generating the in vitro release profile requires five days or less and the predicted drug release profile extends to at least 200 days. In various embodiments, the step of generating the in vitro release profile requires five days or less and the predicted drug release profile extends to at least 250 days.

In various embodiments, the method comprises determining a concentration of the compound in the fluid. In various embodiments, the step of generating the depot comprises initiating a fluid solidification process. In various embodiments, the step of generating the depot comprises injecting the drug formulation into the scaffold during the solidification process. In various embodiments, the step of generating the depot comprises completing the fluid solidification process.

In various embodiments, the injecting step uses an autoinjector. In various embodiments, the injecting step uses a manual injection method.

In various embodiments, the scaffold includes a depression in a hydrogel and the step of generating a depot by combining a scaffold and a sample comprises depositing the sample into the depression.

In various embodiments, the hydrogel comprises a surface. In various embodiments, the hydrogel comprises a sidewall extending away from the surface and into the hydrogel to a floor forming the depression.

In various embodiments, the method may comprise applying at least one mold to a solidifying solution. In various embodiments, the mold includes at least one protrusion extending into the solidifying solution. In various embodiments, the mold includes three protrusions. In various embodiments, the recess created by the protrusion may be able to receive about 6 ul volume of formulation. In various embodiments, the method may comprise solidifying the solidifying solution into a hydrogel. In various embodiments, the method may comprise removing the mold to generate the depression. In various embodiments, the protrusion may be cylindrical. In various embodiments, wherein the depression may be conical

In various embodiments, the solidifying solution may comprise between 0.5% to 2% agarose. In various embodiments, the solidifying solution may comprise a phosphate buffered saline at pH 7.4.

In various embodiments, the step of generating the depot further comprises diffusing an excipient from the combined injection formulation and into the scaffold.

In various embodiments, the characteristic of the fluid may include a drug compound concentration. In various embodiments, the characteristic of the fluid may include a drug analyte. In various embodiments, the drug analyte may include a salt. In various embodiments, the drug analyte may include a prodrug.

In various embodiments, the method further comprises the step of cleaving the prodrug using an enzyme. In various embodiments, the enzyme may be associated with the fluid. In various embodiments, the enzyme may be associated with the scaffold.

In various embodiments, the drug analyte may include a reactionary product of the drug compound. In various embodiments, the characteristic of the fluid includes an opacity. In various embodiments, the characteristic of the fluid includes a pH.

In various embodiments, the method further comprises mixing the fluid. In various embodiments, the mixing may be continuous. In various embodiments, the mixing may be intermittent.

In various embodiments, the characteristic of the fluid includes a free acid. In various embodiments, the characteristic of the fluid includes a free base.

### FIGURES

FIG. 1 illustrates a system for predicting a drug release profile of a long-acting injectable formulations in accordance with various embodiments.

FIG. 2 illustrates a sample receiver for a system for predicting a drug release profile of a long-acting injectable formulations in accordance with various embodiments.

FIG. 3 is a system for predicting a drug release profile of a long-acting injectable formulations in accordance with various embodiments.

FIG. 4 is a system for predicting a drug release profile of a long-acting injectable formulations in accordance with various embodiments.

FIG. 5 is a system for predicting a drug release profile of a long-acting injectable formulations in accordance with various embodiments.

FIG. 6 is a schematic diagram of a computer system adaptable for use in or as part of the various technologies described herein in accordance with various embodiments.

FIG. 7 is a schematic diagram of an exemplary process for testing new long-acting injectable formulations according to various embodiments.

FIG. 8 is a method for predicting a drug release profile of a long-acting injectable formulation according to various embodiments.

FIG. 9 is a method analyzing a fluid at a plurality of timepoints according to various embodiments.

FIG. 10 illustrates a process using a mold to generate a sample receiver site according to various embodiments.

FIG. 11 illustrates a process for generating a depot from a long-acting injectable formulation according to various embodiments.

FIG. 12 photographically illustrates ex vivo pig skin depot formation for comparison to in vitro agarose hydrogel depot formations shown in FIG. 13. FIG. 12, panels A and C show ex vivo pig skin prior to depot formation and FIG. 12, panels B and D show depot formation occurring after one hour.

FIG. 13 photographically illustrates in vitro agarose hydrogel well formation for comparison to ex vivo pig skin depot formations shown in FIG. 12. FIG. 13, panels A, B, and C show in vitro depot formation, partially precipitated over 30 minutes. FIG. 13, panels D, E, and F show in vitro depot formation, completed precipitated over 30 minutes.

FIG. 14 schematically illustrates data showing optimized physical parameters of a depression or well for receiving a long-acting injectable formulation, according to various embodiments.

FIG. 15 is experimental data showing in vitro absorption profiles of four solution-based subcutaneous long-acting injectable formulations.

FIG. 16 is experimental data showing in vivo release profiles of four solution-based subcutaneous long-acting injectable formulations.

FIG. 17 is real data that was collected over five days using our in vitro assay (panel A) and was then processed using historically collected data (panel B) to generate a predictive model that has been time-scaled from five days to 250 days (panel C).

FIG. 18 is experimental data showing use of convolution of time-scaled in vitro release profile data (panel A) with unit input response (UIR) obtained using an intravenous bolus (panel B) to generate predictive release profiles (panel C).

FIG. 19, panel A is experimental data showing mean in vitro release profiles of subcutaneous solution versus suspension long acting injectables of API. FIG. 18, panel B is experimental data showing mean deconvoluted in vivo absorption profiles of subcutaneous solution versus suspension long acting injectables of API.

FIG. 20 is experimental data comparing low dose and high dose formulations in vitro (panel A) and in vivo (panel B).

FIG. 21, panel A shows a depot configuration for the low dose in vitro assay and FIG. 20, panel B shows a depot configuration for the high dose in vitro assay of FIG. 19.

### DETAILED DESCRIPTION

The present disclosure provides insights and technologies useful in accelerating development of long-acting injectable drug formulations. More specifically, the technologies described herein address the unmet need of generating predictive results of a long-acting injectable drug formulation in time that are shorter than the proposed useful medical life of the injected medication. Such technology incorporates the use of both wet lab and analytical systems to generate predictive drug release profiles for patients.

Technologies testing long-acting injectable drug formulations are described herein. Technologies providing for analytical tools increasing biopredictive accuracy over long times are provided herein. In the figures, numerous specific details are set forth to provide a thorough understanding of certain embodiments. A skilled artisan will appreciate that the systems and methods described herein may be used in a variety of ways and circumstances that are not limited to what is specifically detailed. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences can be varied and still remain within the spirit and scope of certain embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

### I. Definitions

In order that the present disclosure may be more readily understood, certain terms are first defined. As used in this application, except as otherwise expressly provided herein, each of the following terms shall have the meaning set forth below. Additional definitions are set forth throughout the application. The headings provided herein are not limitations of the various aspects of the disclosure, which aspects should be understood by reference to the specification as a whole.

As used herein, the term the terms “a” and “an” are used per standard convention and mean one or more, unless context dictates otherwise.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2^{nd} ed., (2001), CRC Press; “The Dictionary of Cell & Molecular Biology”, 5^{th} ed., (2013), Academic Press; and “The Oxford Dictionary Of Biochemistry And Molecular Biology”, Cammack et al. eds., 2^{nd} ed, (2006), Oxford University Press, provide those of skill in the art with a general dictionary for many of the terms used in this disclosure.

As used herein, the term “and/or” is to be understood as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or,” as used in a phrase such as ‘A, B, and/or C” is intended to encompass each of the following aspects: A, B, and C; A, B, or C; A or C; A or B; B or C; A and C; A and B; B and C; A (alone); B (alone); and C (alone).

As used herein, the term the use of the alternative (e.g., “or”) should be understood to mean either one, both, or any combination thereof of the alternatives.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

As used throughout, the term “likely” refers to having a higher probability of occurring than not, or alternatively, of having a higher probability of occurring versus a predetermined control of average. By way of non-limiting example, a patient likely to experience toxicity following a cell therapy refers to that patient having a higher probability of experiencing toxicity than not. Alternatively, a patient likely to experience toxicity following a cell therapy refers to that patient having a higher statistical chance of experiencing toxicity as compared to the average occurrence of toxicity in a patient population treated with the cell therapy. One of ordinary skill in the art would recognize additional definitions in addition to the aforementioned.

As used herein, the terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

As used herein, the terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

As used herein, the terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

### II. Exemplary Systems for Predicting a Drug Release Profile of Long-Acting Injectable

Exemplary systems for predicting a drug release profile of a long-acting injectable are described herein.

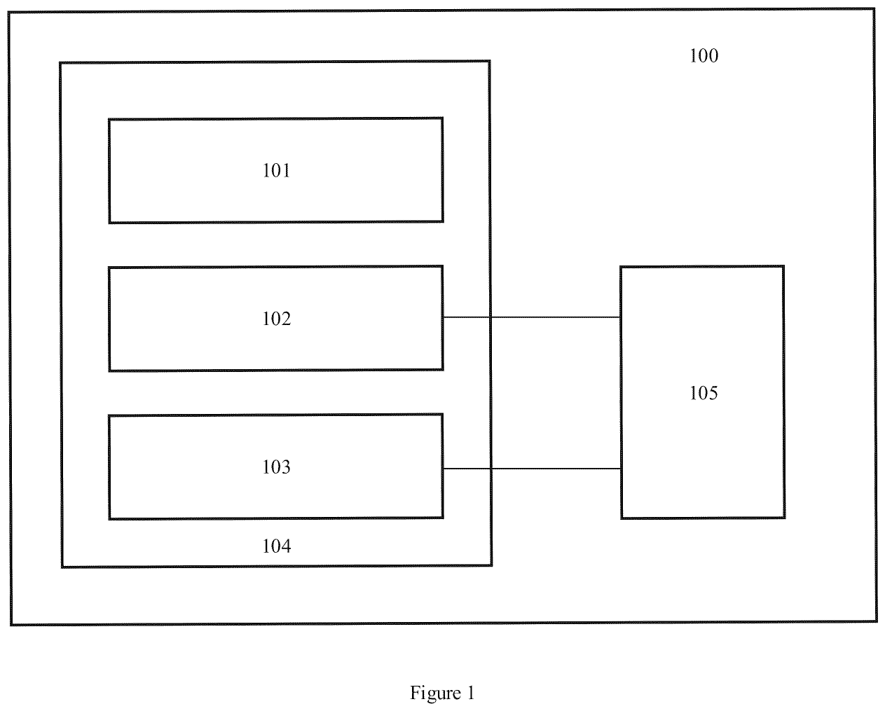

FIG. 1 illustrates a system for predicting a drug release profile of a long-acting injectable formulation 100 in accordance with various embodiments. In various embodiments, the system may comprise a sample receiver 101, a mixing element 102, a sensor system 103, a chamber 104, and/or a computer system 105.

In various embodiments, all or a portion of the sample receiver 101 may be disposed within the chamber 104. In various embodiments, the sample receiver 101 may be fluidically enclosed in the chamber 104. For example, the chamber 104 may serve as a housing for a fluid (e.g., a release fluid). In various embodiments, the sample receiver 101 may be submerged in the fluid contained within the chamber 104. In various embodiments, the fluid may serve as a medium for acting similarly to an extra cellular matrix. For example, a drug compound or another molecule from the sample receiver 101 may be release into the surrounding environment (e.g., a fluid having properties similar to those of an extracellular fluid and/or extracellular matrix for the purposes of drug release into a biological organism such as a human patient).

More specifically, a sample (e.g., a solution or mixture including at least a drug compound and an excipient) may be released, or a portion or constituent thereof, into a fluid according to various embodiments. In various embodiments, the sample, drug compound, drug analyte, or some other compound described here or elsewhere may be detectable using a sensor system 103.

In various embodiments, a mixing system 102 may include a mixing element. In various embodiments, all or a portion of the mixing element 102 may be disposed within the chamber 104. Various commercial mixing systems may be adapted for use with the technologies described herein. In various embodiments, one or more mechanical and/or method of use elements may need to be customized to meet one or more operating requirements.

In various embodiments, a mixing element 102 may include a blade designed for mixing fluids. In various embodiments, a mixing element may include a paddle for mixing fluids. In various embodiments, a mixing element 102 may include an impeller designed for mixing fluids.

In various embodiments, a mixing element 102 may be specifically selected for the purpose of mixing a drug release medium based on a functional and/or physical characteristic. A variety of commercially available mixing elements may be suitable for mixing a fluid such as a drug release medium.

In various embodiments, a sensor system 103 may be used to detect, measure, and/or quantify contaminants within the fluid (e.g., release fluid). In various embodiments, all or a portion of the sensor system 103 may be disposed within the chamber 104. In various embodiments, the sensor system 103 may detect a quality or characteristic of the fluid.

In various embodiments, the sensor system 103 may detect a pH. In various embodiments, the sensor system 103 may detect a temperature. In various embodiments, the sensor system 103 may detect an opacity. In various embodiments, the sensor system 103 may detect a label. For example, a label may be attached to a biologic molecule (e.g., a nucleic acid, an amino acid). In various embodiments, labels may have specificity for a specific molecule. In various embodiments, labels may be specific for a drug compound and/or constituent thereof. In various embodiments, labels may be specific for an excipient or an ingredient of the excipient. In various embodiments, labels may associate with any molecule being release from a depot into a surrounding fluid for analysis. In various embodiments, a label may have specificity for Lenacapavir.

In various embodiments, a chamber 104 may hold a fluid (e.g., a release fluid to be analyzed). In various embodiments, the chamber 104 may function serve as a location for contacting a sample receiver 101 with the fluid. For example, in various embodiments the sample receiver 101 may include a depot which may include a drug compound and additional molecules. In various embodiments, the drug compound and or the additional molecules may leech ingo the fluid. The fluid may then be analyzed over two or more timepoints to generate a drug release profile.

In various embodiments, a computer system 105 may be in electronic communication with a mixing system 102. For example, the computer system 105 may control the operation of the mixing system 102. In various embodiments, an optimized mixing profile may be achieved by adjusting the speed of a mixing system.

In various embodiments, the computer 105 may also be in electronic communication with a sensor system 103. In various embodiments, one or more of the characteristics or qualities being detected or measured by the sensor system 103 may be affected by fluid flow rate. In such embodiments, the computer 105 may direct the mixing system to adjust an operating parameter (e.g., impeller rotational rate, paddle reciprocating rate, etc.)

In various embodiments, a variety of commercially available seals, connectors, and other components may be used in the system for predicting a drug release profile of a long-acting injectable. For example, the system includes a wet lab system that is being controlled and analyzed by a computer system. As such, in various embodiments, the computer system 105 will have electronic access to the interior of the chamber 104.

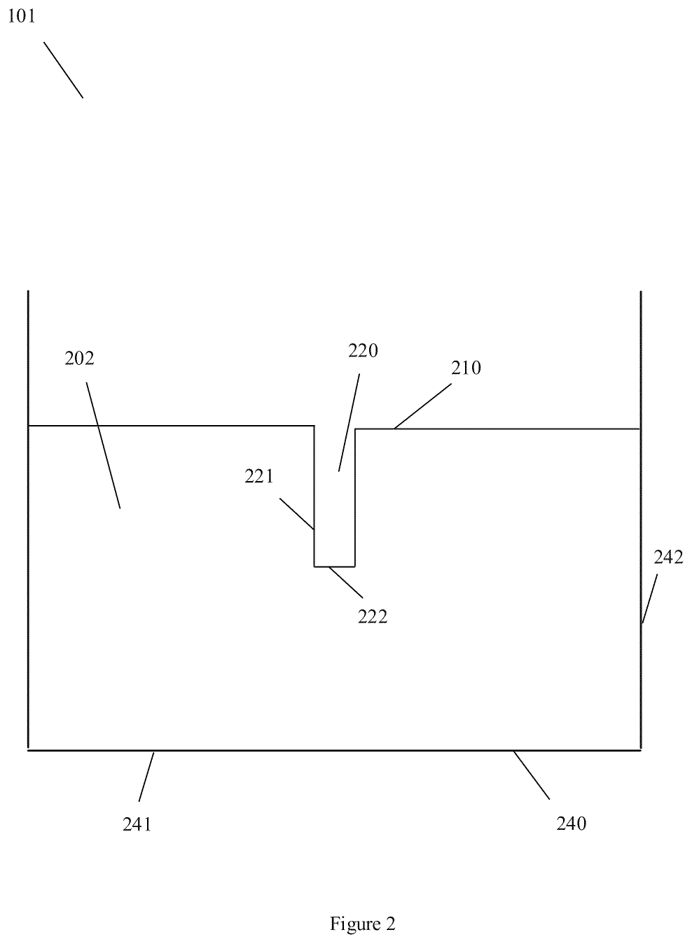

FIG. 2 illustrates a sample receiver 101 for a system for predicting a drug release profile of a long-acting injectable 100 in accordance with various embodiments. In various embodiments, the sample receiver 101 may be disposed within the chamber 104.

In various embodiments, the sample receiver 101 may comprise a structural support 240 for supporting a scaffold 202 (e.g., a hydrogel) and a depression 220 (e.g., a well) within the scaffold 202.

In various embodiments, the structural support 240 may provide lateral support to the scaffold 202. In various embodiments, the support structure 240 may prevent scaffolds 202 from collapsing. A non-limiting example of a structural supports 240 may include an open ended container. In various embodiments, the structural support 240 may surround or partially surround the scaffold 202. A non-limiting example of a structural support 240 may include a heat resistant container.

In various embodiments, a structural support 240 may comprise a container, a receptacle, a vessel, a canister, a drum, a cassette, a well, a cup, a box, and/or a receptible having at least one open end.

In various embodiments, a scaffold 202 may be situated within a structural support 240. In various embodiments, the scaffold 202 may be formed by pouring a hot material into the structural support 240 and allowing the hot material to cool and solidify. In various embodiments, a structural support 240 may include a floor 241 connected to an upstanding sidewall 242.

In various embodiments, a scaffold 202 may include a depression 220. In various embodiments, a depression may be designed to accept a sample (e.g. a formulation comprising a drug compound and an excipient). In various embodiments, the depression 220 may include a volume equal to that of the sample.

In various embodiments, a scaffold 202 designed for use in a system for predicting a drug release profile of a long-acting injectable may comprise a surface 210 and a depression sidewall 221. In various embodiments, a depression 220 may include a surface 210, a depression sidewall 221, and a depression floor 222. In various embodiments, the depression sidewall 221 may extend away from the surface 210 and into the scaffold 202 to a floor 222 to form the depression 220. In various embodiments, the depression 220 may include a cylindrical shape. In various embodiments, the depression 220 may include a rectangular shape. In various embodiments, the depression 220 may include a conical shape. In various embodiments, patterns may be included on the sidewall 221 and/or floor 222 to increase the surface area. In various embodiments, patterns may be included on the sidewall 221 and/or floor 222 to decrease the surface area.

In various embodiments, the scaffold 202 may comprise a hydrogel. In various embodiments the hydrogel may comprise between about 0.5% to 2% agarose. In various embodiments, the hydrogel may comprise a phosphate buffered saline at pH 7.4.

In various embodiments, the structural support 240 includes a floor 241 having at least one upstanding sidewall 242 for supporting the hydrogel 202. In various embodiments, the structural support 240 may include a container, receptacle, vessel, canister, drum, cassette, well, cup, box, or some other device suitable for receiving a hot agar solution and having at least one open end.

In various embodiments, a structural support 240 may serve to receive a hot solution. As such, exemplary materials for constructing a structural support 240 may include aluminum or temperature treated glass in accordance with various embodiments. In various embodiments, the materials may be selected for having low leachability so as not to interfere with the detection and measurement readings taken from the fluid.

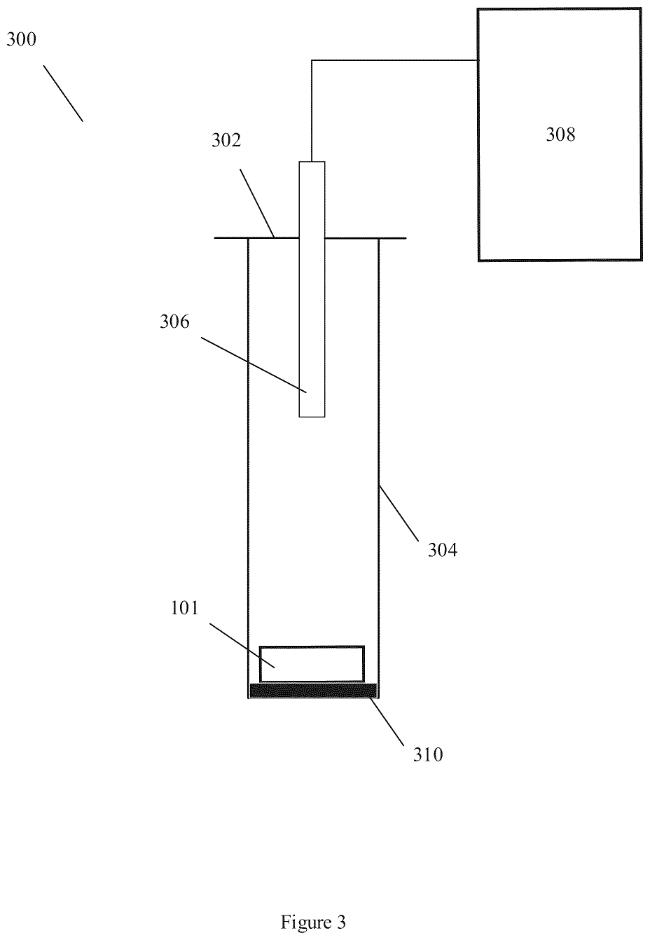

FIG. 3 is a system for predicting a drug release profile of a long-acting injectable 300 in accordance with various embodiments.

In various embodiments, the system for predicting a drug release profile of a long-acting injectable 300 may comprise a chamber 304. In various embodiments, the chamber 304 may be adapted for holding a fluid. In various embodiments, the fluid may serve to receive analytes being released from a sample receiver 101.

In various embodiments, the chamber 304 may house the sample receiver 101. In various embodiments, the sample receiver 101 may be suspended within the chamber 304 so that fluid surrounds or partially surrounds the sample receiver 101. In various embodiments, an adapter may be used to secure the sample receiver 101 within the chamber 304. For example, the adapter may interact with the chamber 304 and the sample receiver 101 in a way that prevents relative movement between the two. In various embodiments, the adapter may rely on a frictional force between a chamber 304 or a portion thereof and the adapter or a portion thereof. In other and additional embodiments, a portion of the chamber 304 wall may be adapted to interact with the adapter. In various embodiments, a groove or a depression may be configured to receive a portion of the adapter. In various embodiments, a fastening device such as screws, pins, adhesive, weld, or any other known fastening method may be used to secure the adapter to the chamber 304.

In various embodiments, the mixing system my comprise a mixing element 310. In various embodiments, a mixing element 310 may comprise a stir bar. In various embodiments, the mixing element 310 may serve to mix the fluid (e.g., a release fluid) held by the chamber 304. In various embodiments, the speed of the mixing element 310 may be adjustable. In various embodiments, a computer may be responsible for change a motor speed, wherein a motor drives the mixing element 310. In various embodiments, the stir bar may be driven by a magnetic plate (now shown).

In various embodiments, a mixing element 310 may take any form capable of moving fluid within the chamber at appropriate mixing rates to help mimic extracellular fluid. For example, in various embodiments, the mixing element 310 may be a paddle mixer.

In various embodiments, the mixing element 310 may be driven by a motor. In various embodiments, one or more gears may be used to reorient the directional force. For example, an impeller requires rotational force. For another example, a motor may provide reciprocating force to a paddle mixing element 310.

In various embodiments, the in vitro release assay and systems described herein serve the purpose of detecting and/or measuring a compound being release from a sample receiver 101 and into a fluid within a chamber 310. By measuring the concentration of the compound over time an in vitro drug release profile may be generated according to various embodiments. In various embodiments, the measurements may be compared to a reference to generate a predicted in vivo drug release assay.

In various embodiments, a compound being detected or measured may include a drug compound (e.g., Lenacapavir), a reactionary product from the drug compound, an ingredient from an excipient, or any other compound in the sample or capable or being generated by the sample through a reaction, chemical or otherwise. In various embodiments, multiple compounds may be detected and/or measured at once contributing to a measurement considering the general fluid environment (e.g., opacity, pH, etc.).

In various embodiments, a probe 306 may be used to detect or measure the compound. In various embodiments, the probe 306 may detect and/or measure a pH of the fluid. In various embodiments, the probe 306 may detect and/or measure an opacity of the fluid. In various embodiments, the probe 306 may detect and/or measure a concentration of a specific molecule (e.g., detection of a labeled compound).

In various embodiments, a computer system 308 may be used for a variety of purposes such as controlling the mixing element 310 operational settings to control fluid mixing parameters. For example, a release assay may require specific fluid flow rates within a chamber 304. In such embodiments, the computer system 308 may be preprogrammed with operational parameters specific for the release assay.

In various embodiments, the computer system 308 may be used to communicate (e.g., via wired and/or wireless communication) with the probe 306. In various embodiments, the computer system 308 may include a storage component (e.g., a hard drive, a cloud storage, etc.) that may store measurements taken by the probe 306. In various embodiments, the computer system 308 functions primarily as an instrument controller and may send data to a separate computer system.

In various embodiments, a computer system 308 may function to control a feedback loop using the probe 306 and the mixing element 310. For example, depending on what the probe 306 detects or measures, the computer system 308 may use that information to either increase or decrease a mixing rate of the fluid within the chamber 304 by adjusting a rate of speed of the mixing element 310.

In other embodiments, the mixing speeds may be static and the computer system 308 may serve to turn hardware on or off and to receiver and store data.

In various embodiments, the system for predicting a drug release profile of a long-acting injectable 300 may include a lid 302 for sealing the chamber 304.

In various embodiments, the lid 302 may provide access to the chamber 304. For example, fluid may enter through the lid 302. In various embodiments, the lid 302 may be partially or completely removed so that a technician may complete maintenance on the system 300. In various embodiments, the lid 302 may be partially or completely removed so that a sample receiver 101 may be positioned within the chamber 304.

In various embodiments, the probe 306 may extend through an opening in the lid 302. In various embodiments, the probe 306 may be permanently attached to the lid 302 (e.g., by weld or solid composite housing). In various embodiments, the probe 306 may separate and/or separatable from the lid 302. In various embodiments, different probe 306 types may be used depending on an analyte being detected. Non-limiting examples of characteristics being probe 306 may include pH, opacity, florescence, etc. In various embodiments, the probe 306 may be designed to make direct contact with the fluid in the chamber 304.

In various embodiments, the probe 306 may extend through the lid 302. In various embodiments, the probe may be at least partially disposed within the chamber. In various embodiments, the probe comprises a fiber optic ultraviolet (UV) probe.

In various embodiments, the lid 302 may affix to the chamber 304 such that the fluid within the chamber is environmentally sealed (e.g., hermetically sealed). In various embodiments, the opening through which the probe 306 extends through the lid 302 also creates a seal. In various embodiments, the seals between the chamber 304, lid 302, probe 306 may be water resistant. In various embodiments, the seals between the chamber 304, lid 302, probe 306 may prevent fluid flow between the chamber 304 and external environment.

In various embodiments, the lid 302 may include a sampling port. In various embodiments, the lid 302 may include a gas exchange port. In various embodiments, the lid 302 may include any kind of port suitable for exchanging any material.

In various embodiments, the chamber 304 may comprise a distal end and a proximal end. In various embodiments, the chamber 304 may further comprise a lid 302 for sealing the chamber 304 at the proximal end and the sample receiver 101 disposed at the distal end.

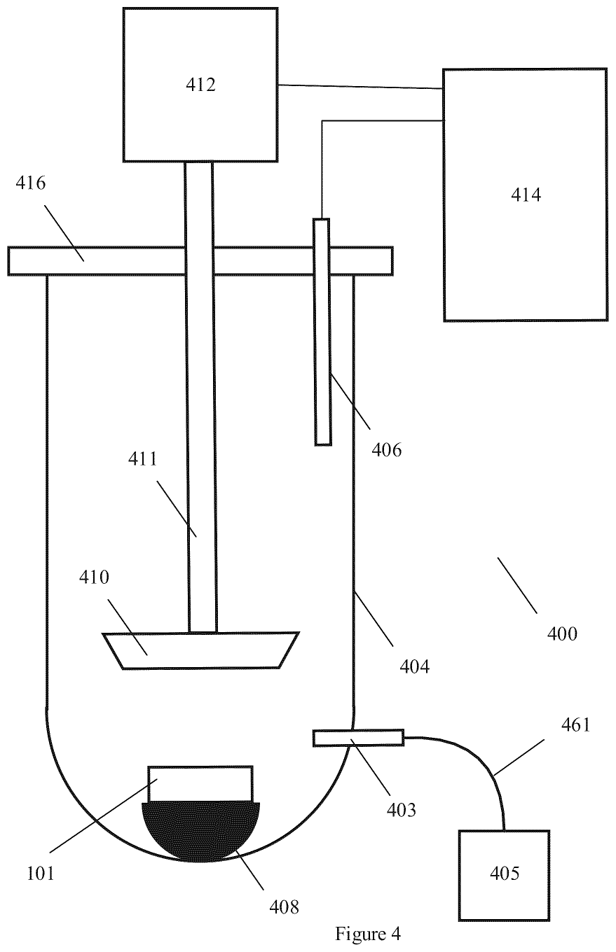

FIG. 4 is a system for predicting a drug release profile of a long-acting injectable 400 in accordance with various embodiments. In various embodiments, the system for predicting a drug release profile of a long-acting injectable 400 may comprise a chamber 404 for receiving a fluid, a sample receiver 101 disposed within the chamber 404, a mixing system for mixing the fluid, a probe 406 for detecting a characteristic of the fluid, and a lid 416 for enclosing the chamber 304.

In various embodiments, the chamber 404 may hold a fluid when the system is in use. In various embodiments, the chamber 404 may include a rounded bottom and an opposing opening. In various embodiments, a lid 416 may seal the opposing opening.

In various embodiments, the chamber 404 may include a rigid material. In various embodiments, the chamber 404 may include a flexible material. In various embodiments, the chamber 404 is designed to have one or more access points for probes 406, driveshafts 411, fluid input and outlet, etc. In various embodiments, the one or more access points may be sealed such that fluid exchange between the chamber and external environment is either prevented or reduced. For example, several commercially available seals exist that may allow water-tight insertion of a drive shaft 411 through a chamber 404 allowing transfer of rotational or reciprocating energy.

In various embodiments, a sample port 103 may provide fluidic access to the chamber 404. For example, a portion of the fluid within the chamber 404 may be withdrawn via the sample port 403 for off-line analysis. In various embodiments, off-line analysis allows for modularity of the system 440 because a wide array of assay may be performed on the sample. Additionally, such an embodiment may allow labs to use existing analytical tools within their lab space. In various embodiments, a sample port 403 may be fluidically connected to a fluid collection apparatus 405.

In various embodiments, the sample port 403 may provide access to the chamber 404 while the system 400 is in operation. In accordance with various embodiments, a sample may be first associated with a scaffold of a sample receiver 101. In various embodiments, the sample receiver 101 may then be positioned within the chamber 404. During typical operation, a mixing element 410 may stir and/or agitate a fluid while fluid samples may be collected at various timepoints. Collection may be completed by drawing fluid from the chamber 404 and through the sample port 403 to a fluid collection apparatus 405. In various embodiments, one or more pumps may be used to pump the fluid out of the sample port 403.

In various embodiments, a pump may be positioned at or a fluidic connection 461.

In various embodiments, the fluid collection apparatus 405 may be removeable for the system 400. Such a configuration may enable ease of off-line analysis.

In various embodiments, a fluid may be designed to receive a compound (e.g., an analyte) from the sample receiver 101. In various embodiments, the compound being released into the fluid may originate from a depot. In various embodiments, a depot may comprise a precipitate or aggregate formed by reacting a sample with a scaffold (e.g., hydrogels or other physical matrices). In various embodiments, the compound may include a drug compound, a drug related analyte, an excipient or ingredient thereof, or any component or byproduct of a sample or sample processing. In various embodiments, the compound may comprise a reactionary product of the drug compound.

In various embodiments, the mixing system include a motor assembly 412 for driving a mixing element 410. In various embodiments, a driveshaft 411 may be used to transfer power from the motor assembly 412 to the mixing element 410.

In various embodiments, the mixing element 410 may comprise an impeller. In various embodiments, the mixing element 410 may comprise a paddle. In various embodiments, the mixing element 410 may comprise any physical form capable for mixing and/or moving a fluid within the chamber 404.

In various embodiments, a motor assembly 412 may include a motor. In various embodiments, the motor assembly 412 may comprise a gear system for redirecting power (e.g., from rotational to reciprocal).

In various embodiments, a driveshaft 411 may extend through a lid 416. In various embodiments, one or more seals may be used to prevent exchange of fluid between the chamber 404 and external environment. In various embodiments, one or more seals may be designed to interact with the moving driveshaft 411. In various embodiments, the driveshaft 411 may move rotationally. In various embodiments, the driveshaft 411 may move reciprocally.

In various embodiments, the probe 406 may extend through the lid 416 and into the chamber 404 to detect and/or measure a characteristic or quality of the fluid and/or a constitute of the fluid. In various embodiments, a characteristic or quality may include anything that is detectable or quantifiable (e.g., a pH, opacity, fluorescence, labeled molecule, etc.) In various embodiments, the probe 406 may interact directly with the fluid to detect or measure the quality or characteristic of the fluid. In various embodiments, a portion of the probe 406 may interact with the lid 416 to form a seal. In various embodiments, the seal may be water resistant. In various embodiments, the seal may prevent a fluid from being exchanged between the interior of the chamber 404 and an external environment.

In various embodiments, the system may include one or more computer systems 414. The one or more computer systems 414 may be interconnected with any of the components of the system via wired and/or wireless communication or by any other known method. For example, the computer 414 may be in wired electronic communication and/or wireless electronic communication with the motor assembly 412. In various embodiments, the computer may direct the motor assembly to adjust motor speed. In various embodiments, adjustments may be static and predetermined. In various embodiments, adjustments may be made in real time. In various embodiments, the computer system 414 may analyze probe 406 data to determine an adjustment to motor speed in real time. In various embodiments, the computer 414 may send electronic signals to the motor assembly 412 selecting a static setting for motor speed. For example, it is possible that adjusting mixing element 410 speed over time could account for changing conditions at or near and injection site. As an alternate example, consistent mixing element 410 rate may be desirable in various embodiments.

In various embodiments, a release assay may require specific fluid flow rates within a chamber 404. For example, different formulations tested may require different flow rate selection. Selection may be determined by the proposed in vivo injection site location. In such embodiments, the computer system 414 may be preprogrammed with operational parameters specific for the release assay.

In various embodiments, the computer system 414 may be used to communicate with the probe 406. In various embodiments, the computer system 414 may include a storage component (e.g., a hard drive, a cloud storage, etc.) that may store measurements taken by the probe 406.

In various embodiments, a computer system 414 may function to control a feedback loop using the probe 406 and the mixing element 410. For example, depending on what (e.g., analyte[s]) the probe 406 detects or measures, the computer system 414 may use that information to either increase or decrease a mixing rate of the fluid within the chamber 404 by adjusting a rate of speed of the mixing element 410.

In various embodiments, the chamber 404 may house the sample receiver 101. In various embodiments, the sample receiver 101 may be disposed within and/or suspended in the chamber 404. In various embodiments, a fluid may surround partially surround the sample receiver 101. In various embodiments, an adapter 408 may be used to secure the sample receiver 101 to a location within the chamber 404. For example, an adapter 408 may interact with the chamber 404 and the sample receiver 101 in a way that prevents relative movement between the chamber 404 and the sample receiver 101. In various embodiments, the adapter 408 may rely on a frictional force between a chamber wall and a portion of the adapter. In other and additional embodiments, a portion of the chamber 404 wall may be adapted to interact with the adapter 408. In various embodiments, a groove or a depression may be configured to receive a portion of the adapter 408. In various embodiments, a fastening device such as screws, pins, adhesive, weld, or any other known fastening method may be used to secure the adapter to the chamber 404 and/or the sample receiver 101.

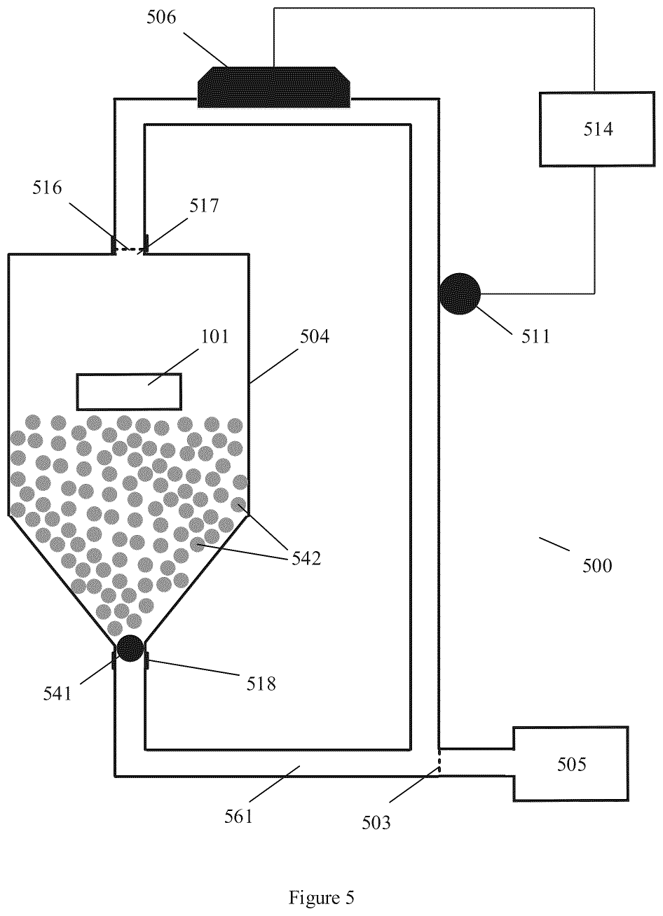

FIG. 5 is a system for predicting a drug release profile of a long-acting injectable 500 in accordance with various embodiments. In various embodiments, a system for predicting a drug release profile of a long-acting injectable 500 may comprise a chamber 504 for receiving a fluid, a sample receiver 101 disposed within the chamber 504, and a mixing system 511 for mixing the fluid within the chamber 504.

In various embodiments, the sample receiver 101 may comprise a structural support for supporting a scaffold and a sample receiver site associated with the scaffold.

In various embodiments, the sample receiving site may be located within an interior of the scaffold. In various embodiments, the sample receiving site may comprise a surface of the scaffold. In various embodiments, the sample receiving site may comprise a depression associated with the scaffold.

In various embodiments, the depression may comprise a sidewall extending into a surface to a floor. In various embodiments, the sidewall and floor form the depression. In various embodiments, the depression may comprise a cylindrical shape. In various embodiments, the depression may comprise a conical shape.

In various embodiments, the scaffold may comprise a hydrogel. In various embodiments, the hydrogel may comprise between about 0.5% to about 2% agarose. In various embodiments, the hydrogel may comprise a phosphate buffered saline. In various embodiments, the pH of the phosphate buffered saline may include pH 7.4.

In various embodiments, components may be added and/or used in the creation of a hydrogel to mimic living tissue. Non-limiting examples of living tissue may include intramuscular tissue and subcutaneous tissue. In various embodiments, the hydrogel may comprise collagen. In various embodiments, the hydrogel may comprise polyacrylamide.

In various embodiments, the scaffold may comprise albumen.

In various embodiments, the scaffold may comprise at one enzyme.

In various embodiments, the chamber 504 may comprise a sample port 503. In various embodiments, the sample port 503 may be fluidically connected to a fluid sample collection apparatus 505.

In various embodiments, system for predicting a drug release profile of a long-acting injectable 500 may comprise a lid 516 for sealing the chamber 504 at an inlet 517 (e.g., an opening).

In various embodiments, system for predicting a drug release profile of a long-acting injectable 500 may comprise a probe 506. In various embodiments, the probe 506 may be positioned at any point at or near a fluidic connection 561. In various embodiments, a probe 506 may be located at an exterior of the fluidic connection 561 and the fluidic connection 561 may comprise a transparent material.

In various embodiments, a fluidic connection 561 may comprise any device capable for channeling a fluid. Non-limiting examples of fluidic connections 561 may include tubing, pipes, etc. In various embodiments, fluidic connections 561 may comprise a polymer.

In various embodiments, the probe 506 may comprise a fiber optic ultraviolet (UV) probe.

In various embodiments, the chamber 504 may further comprise an inlet 517 for transferring the fluid into the chamber 504. In various embodiments, the chamber 504 may further comprise an outlet for transferring the fluid out of the chamber.

While in operation a mixing system 511 may be used to circulate a fluid out of the chamber 504 thought an outlet 518 and back into the chamber 504 through an inlet 517. Various methods described herein include one or more steps where a fluid circulates. In various embodiments, circulation may help to mimic conditions of an in vivo system.

In various embodiments, a mixing system 511 may comprise one or more pumps. In various embodiments, the one or more pumps may be positioned at or along a position of a fluidic connection 561. In various embodiments, a fluidic connection 561 may comprise a flexible polymer tube which can be manipulated using a peristaltic pump. In various embodiments, the fluidic connection 561 may comprise a rigid material.

In various embodiments, a bead situated adjacent to the outlet 518. In various embodiments, the bead 541 may prevent backflow. In various embodiments, the bead may be movable by fluid flow out of the chamber 504. In various embodiments, backflow may cause the bead 541 to obstruct the outlet 518.

In various embodiments, the sample receiver 101 may be positioned between the inlet 517 and the outlet 518. In various embodiments, a space within the chamber 504 and between the sample receiver 101 and the outlet 518 may be at least partially filled with a solid matrix 542 to support the sample receiver 101. In various embodiments, the solid matrix 542 comprises a plurality of glass beads.

In various embodiments, the system for predicting a drug release profile of a long-acting injectable 500 may comprise a fluid sample collection apparatus 505 fluidically connected to the outlet 518 by a fluidic connection 561. In various embodiments, the fluid sample collection apparatus 505 may be detachable from the fluidic connection for off-line analysis of the fluid.

In various embodiments, the system for predicting a drug release profile of a long-acting injectable 500 may comprise a probe 506 positioned at or near a fluidic connection 561.

In various embodiments, the probe 506 may measure a quality of the fluid. Non-limiting examples of qualities may include opacity, pH, and fluorescence.

In various embodiments, the fluid may include a biorelevant media. In various embodiments, the biorelevant media comprises between 2 to 4 percent bovine or human serum albumin (BSA/HSA). In various embodiments, the fluid further includes 0.02% sodium azide.

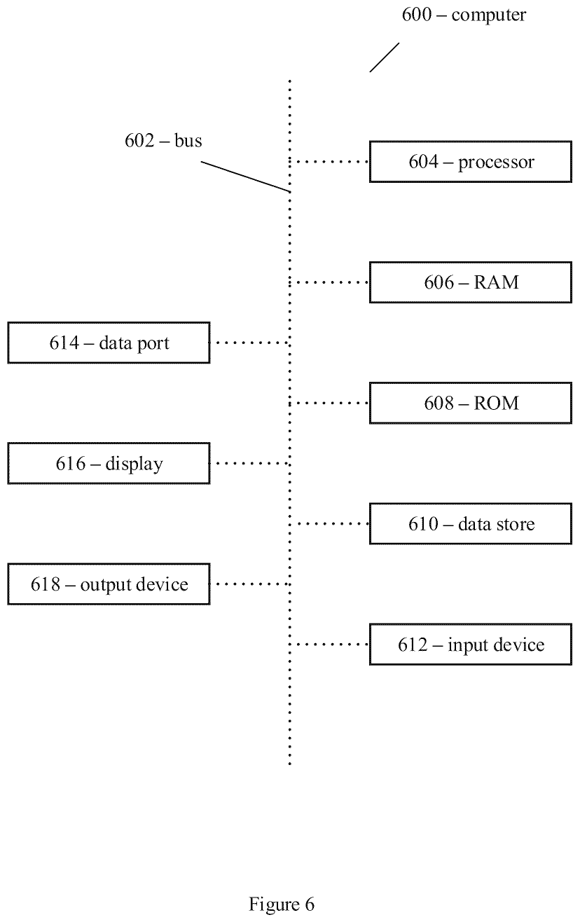

FIG. 6 is a schematic diagram of a computer system 600 in accordance with various embodiments. The computer system 600, upon which embodiments of the present teachings may be implemented. In various embodiments, a computer system 600 may be integrated into other parts of the system (e.g., into computer systems 308, 414, 514). For example, a controller, a data logger, a cell phone tower, a user device, or almost any other aspect of the system may include an integrated computer system 600.

Non-limiting examples of processes that may be carried out by computer system 600 may include operation of devices (e.g., the probes and mixing systems described herein), collection, storage of data taken at two or more timepoints, and processing of probe data used to generate an in vitro release profile of a drug compound from the timepoints. In various embodiments, the computer system 1000 may be responsible for data analysis wherein the data collected at various timepoints and/or the in vitro release profile may be compared to a reference to generate a predicted in vivo release assay. The computer system 1000 may execute on various feedback processes using the hardware described herein. For example, in some assays a mixing system may be controlled in real time wherein adjustments may be made based on probe feedback (i.e., feedback loops).

In various embodiments, the computer system 1000 may control operation of one or more of the various mixing systems described herein. For example, in the various systems presented above there may be a desire to move fluid throughout the chamber to better mimic in vivo systems (e.g., patient tissue such as intramuscular tissue and subcutaneous tissue).

In various embodiments, computer system 1000 may control opening and closing of inlets, outlets, and/or control fluid flow into a fluid sample collection apparatus.

In various embodiments of the present teachings, computer system 600 may include a bus 602 or other communication mechanism for communicating information, and a processor 604 coupled with bus 602 for processing information. In various embodiments, computer system 600 may also include a memory, which can be a random-access memory (RAM) 606 or other dynamic storage device, coupled to bus 602 for determining instructions to be executed by processor 604. Memory also can be used for storing temporary variables or other intermediate information during execution of instructions to be executed by processor 604. In various embodiments, computer system 600 may further include a read only memory (ROM) 608 or other static storage device coupled to bus 602 for storing static information and instructions for processor 604. A data store 610, such as a magnetic disk or optical disk, can be provided and coupled to bus 602 for storing information and instructions.

In various embodiments, an analyte quality (e.g., concentration) may be measured at two or more timepoints by a probe. The quality measurements may be transmitted via electronic communication and stored in memory. In various embodiments, the quality measurements may be further processed by processor 604 to generate an in vitro release profile. In various embodiments, the quality measurements may be used to predict how a drug compound will act in a patient (e.g., length and stability of release pattern). In various embodiments, processor 604 may compare the quality measurements and/or the in vitro release assay to a reference to generate a predicted in vivo release assay. Processor 604 may send the newly generated predicted in vivo release assay to memory to be stored. In various embodiments, the newly generated predicted in vivo release assay may be displayed on a display 616, accessed through data port 614, or externalized through any output device 618 (e.g., digital storage device, paper, etc.).

In some embodiments, computer system 600 can be coupled via bus 602 to a display 616, such as a cathode ray tube (CRT), liquid crystal display (LCD), or light emitting diode display (LED) for displaying information to a computer user. An input device 612, including alphanumeric and other keys, can be coupled to bus 602 for communicating information and command selections to processor 604. Another type of user input device 612 is a cursor control, such as a mouse, a trackball or cursor direction keys for communicating direction information and command selections to processor 604 and for controlling cursor movement on display 616. In various embodiments, the computer system 600 may include a touchscreen display. The input device 612 typically has two degrees of freedom in two axes, a first axis (i.e., x) and a second axis (i.e., y), that allows the device to specify positions in a plane. However, it should be understood that input devices 612 allowing for 3-dimensional (x, y and z) cursor movement are also contemplated herein.

In various embodiments, computer system 600 can be coupled via bus 602 to one or more data ports 614. In various embodiments, the one or more data ports 614 may enable electronic communication between the components via bus 602 of the computer system 600 and other components of the overall system described herein.

Consistent with certain implementations of the present teachings, results can be provided by computer system 600 in response to processor 604 executing one or more sequences of one or more instructions contained in memory 606. Such instructions can be read into memory 606 from another computer-readable medium or computer-readable storage medium, such as a storage device containing information relating to environmental control (e.g., a feedback algorithm) or an environmental condition monitoring system. Execution of the sequences of instructions contained in memory 606 can cause processor 604 to perform the processes described herein. Alternatively, hard-wired circuitry can be used in place of or in combination with software instructions to implement the present teachings. Thus, implementations of the present teachings are not limited to any specific combination of hardware circuitry and software.

According to various embodiments, computer-readable medium (e.g., data store, data storage, etc.) or computer-readable storage medium may comprise any media that participates in providing instructions to processor 604 for execution. Such a medium can take many forms, including but not limited to, non-volatile media, volatile media, and transmission media. Non-limiting examples of non-volatile media can include optical, solid state, and magnetic disks, such as 608. Examples of volatile media can include, but are not limited to, dynamic memory, such as memory 606. Examples of transmission media can include, but are not limited to, coaxial cables, copper wire, and fiber optics, including the wires that comprise bus 602.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 604 of computer system 600 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc. In various embodiments, output devices 1018 such as printers and displays may be used to present output files generated by the processes described herein.

Output devices 618 may be used to communicate real time environmental conditions or a history of environmental conditions. Output device 618 may be used to indicate the condition or one or more metrics associated with the condition of an apheresis material within the system described herein. Output devices 618 may output any processed information.

In various embodiments, output devices 618 may relay information relating to the predictions described herein to an external source.

### III. Exemplary Methods for Predicting a Drug Release Profile of Long-Acting Injectable

The technologies described herein comprise various method embodiments. The methods may be agnostic for use in conjunction with any of the systems described herein according to various embodiments. In various embodiments additional and other embodiments, the methods described herein may be suitable for use on the accompanying systems detailed here.

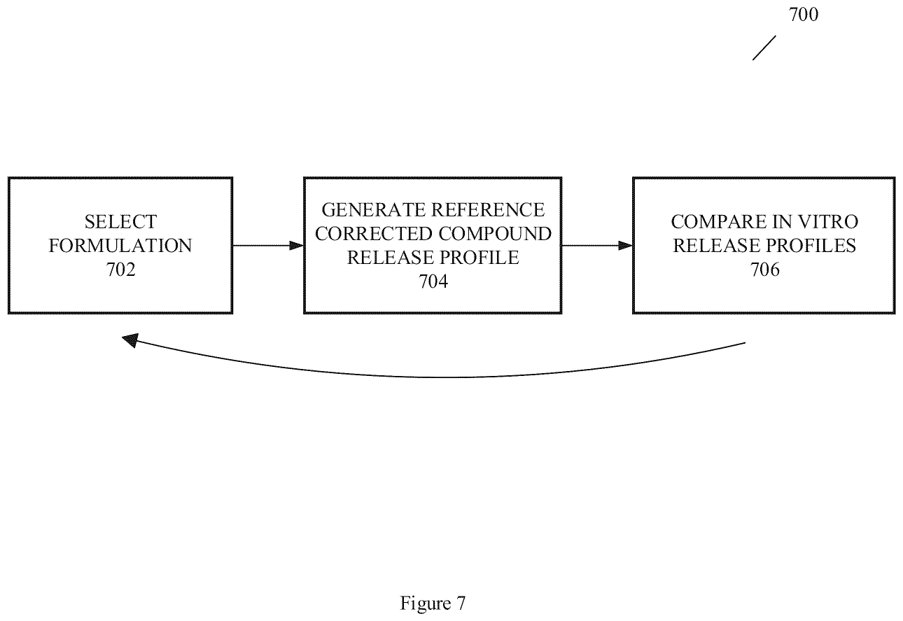

FIG. 7 is a schematic diagram of an exemplary process 700 for testing new long-acting injectable formulations according to various embodiments. One of the challenges in the field as described herein is the long development timelines for long-acting injectable drug formulations. The length of these timelines is compounded by the fact that it may be very helpful to see results of previous studies prior to starting new studies. This remains the case because the performance of past formulations drives the process for determining follow-on formulations.

Step 702 of the process 700 comprises selecting a formulation for a long-acting drug going into an injectable drug administration format according to various embodiments. In various embodiments, the selection process 700 may comprise addition of one or more ingredients. In various embodiments, the selection process 700 may comprise subtraction of one or more ingredients. In various embodiments, the selection process 700 may comprise adjustment of a quantity and/or concentration of an ingredient.

Step 704 of the process 700 comprises generating a reference corrected compound release profile 704 according to various embodiments. In various embodiments, the use of a reference may allow for shortened study periods, thereby, accelerating the development timeline for generating new formulations.

Step 706 of the process 700 comprises comparing in vitro release profiles. In various embodiments, multiple formulations may be tested in a single cohort. In such embodiments, results from each of the cohorts would be compared in step 706 and/or compared to previous cohorts if the data exists. Various comparative statistical methods are known and may be applied to comparative analyses. In various embodiments, the results from step 706 may help determine a new set of one or more formulations to undergo the process 700 again. In various embodiments, the process 700 may be iterative. In various embodiments, the process 700 may be undergo as many cycles as are needed to achieve of desired performance outcome of the long-acting injectable formulation.

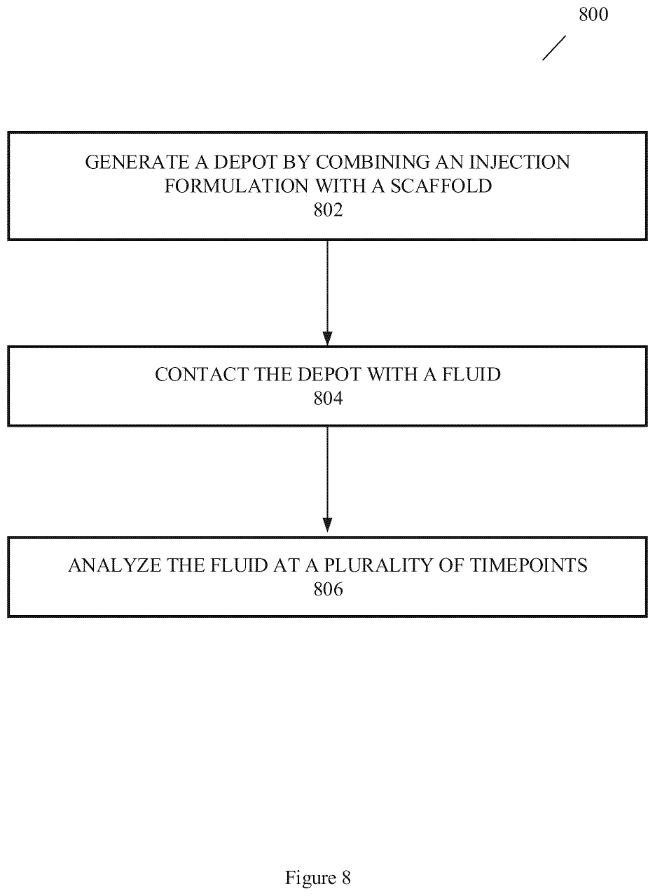

FIG. 8 is a method 800 for predicting a drug release profile of a long-acting injectable formulation according to various embodiments.

In various embodiments, any of the computer systems (e.g., computer system 600) may be used to complete any of the analytical steps of the process 700.

Step 802 of the method 800 comprises generating a depot by combining an injection formulation with a scaffold in accordance with various embodiments. In various embodiments, the injection formulation may comprise a drug compound and an excipient.

Step 804 of the method 800 may comprise contacting the depot with a fluid in accordance with various embodiments. In various embodiments, a compound may diffuse from the depot to the fluid.

Step 806 of the method 800 may comprise analyzing the fluid at a plurality of timepoints.

In various embodiments, a method for predicting a drug release profile for a long-acting injectable formulation may make use of the fact that the characteristic may be influenced by a quantity of the compound in the fluid. In various embodiments, the compound may comprise an analyte that is detectable by a probe.

In various embodiments, the step of generating the in vitro release profile requires less than 30 days and the predicted drug release profile extends to at least 180 days. In various embodiments, the step of generating the in vitro release profile requires five days or less and the predicted drug release profile extends to at least 50 days. In various embodiments, the step of generating the in vitro release profile requires five days or less and the predicted drug release profile extends to at least 100 days. In various embodiments, the step of generating the in vitro release profile requires five days or less and the predicted drug release profile extends to at least 150 days. In various embodiments, the step of generating the in vitro release profile requires five days or less and the predicted drug release profile extends to at least 200 days. In various embodiments, the step of generating the in vitro release profile requires five days or less and the predicted drug release profile extends to at least 250 days.

In various embodiments, the method for predicting a drug release profile of a long-acting injectable formulation further comprises determining a concentration of the compound in the fluid.

In various embodiments, the step of generating the depot may comprise initiating a fluid solidification process. In various embodiments, the step of generating the depot may comprise injecting the drug formulation into the scaffold during the solidification process. In various embodiments, the step of generating the depot may comprise completing the fluid solidification process. In other embodiments, injection may occur after solidification has occurred. In various embodiments, gelling/solidification may occur between 35° and 38° Celsius. In various embodiments, gelling/solidification may occur at room temperature (e.g., approximately 25° Celsius).

In various embodiments, a device may be used to create a well within a scaffold by removing an interior portion of the scaffold. For example, an injection needle (or other device capable for drawing a fluid or solid) may be used to remove the inferior portion to create a well. In various embodiments, an injection formulation may then be injected into the well to form a depot.

In various embodiments, the injecting step may use an autoinjector. In various embodiments, the injecting step may use a manual injection method.

In various embodiments, the scaffold may include a depression in a hydrogel and the step of generating a depot by combining a scaffold and a sample comprises depositing the sample into the depression.

In various embodiments, the hydrogel comprises a surface and a sidewall extending away from the surface and into the hydrogel to a floor forming the depression.

In various embodiments, the method for predicting a drug release profile of a long-acting injectable formulation may further comprise applying at least one mold to a solidifying solution. In various embodiments, the mold may include at least one protrusion extending into the solidifying solution. In various embodiments, the protrusion may be cylindrical.

In various embodiments, the method for predicting a drug release profile of a long-acting injectable formulation may further comprise solidifying the solidifying solution into a hydrogel.

In various embodiments, the method for predicting a drug release profile of a long-acting injectable formulation may further comprise removing the mold to generate the depression.

In various embodiments, the solidifying solution may comprise between 0.5% to 2% agarose. In various embodiments, the solidifying solution may comprise a phosphate buffered saline. In various embodiments, the phosphate buffered saline may be at a pH of 7.4.

In various embodiments, the step of generating the depot may further comprise diffusing an excipient from the combined injection formulation and scaffold.

In various embodiments, the characteristic of the fluid may include a drug compound concentration. In various embodiments, the characteristic of the fluid may include an opacity. In various embodiments, the characteristic of the fluid may include a pH. In various embodiments, the characteristic of the fluid may include ultraviolet light absorption.

In various embodiments, the characteristic of the fluid may include a drug analyte. In various embodiments, the drug analyte may include a salt. In various embodiments, the drug analyte may include a prodrug. In various embodiments, the drug analyte may include an enzyme for cleaving the prodrug. In various embodiments, the drug analyte may include a reactionary product of the drug compound.

In various embodiments, the method for predicting a drug release profile of a long-acting injectable formulation may further comprise mixing the fluid. In various embodiments, the mixing may be continuous. In various embodiments, the mixing may be intermittent.

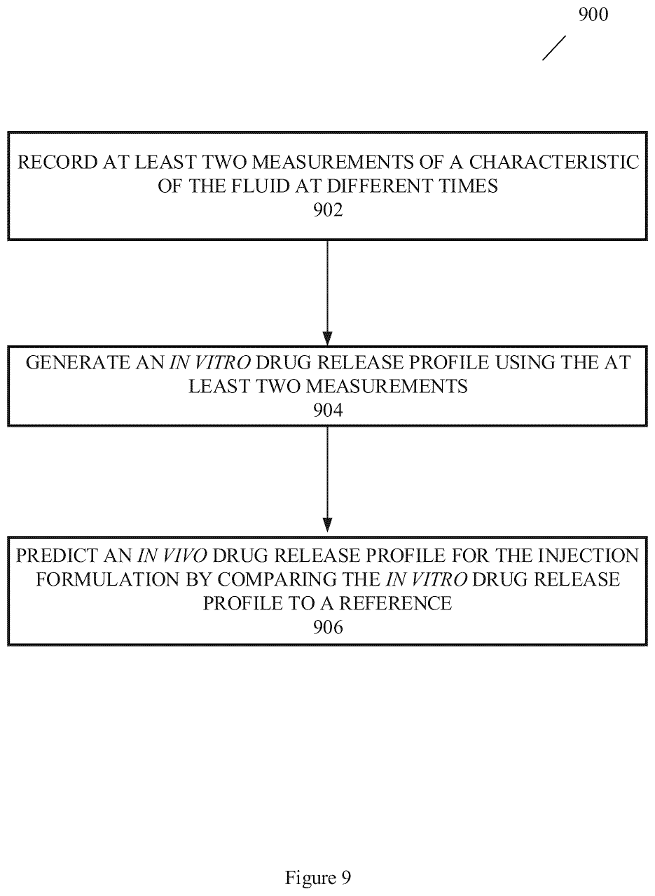

FIG. 9 is a method 900 analyzing a fluid at a plurality of timepoints according to various embodiments. In various embodiments, the process 900 may be completed on one or more of the computer systems described herein.

Step 902 of the method comprises recording at least two measurements of a characteristic of the fluid at different times.

Step 904 of the method comprises generating an in vitro drug release profile using the at least two measurements.

Step 906 comprises predicting an in vivo drug release profile for the injection formulation by comparing the in vitro drug release profile to a reference.

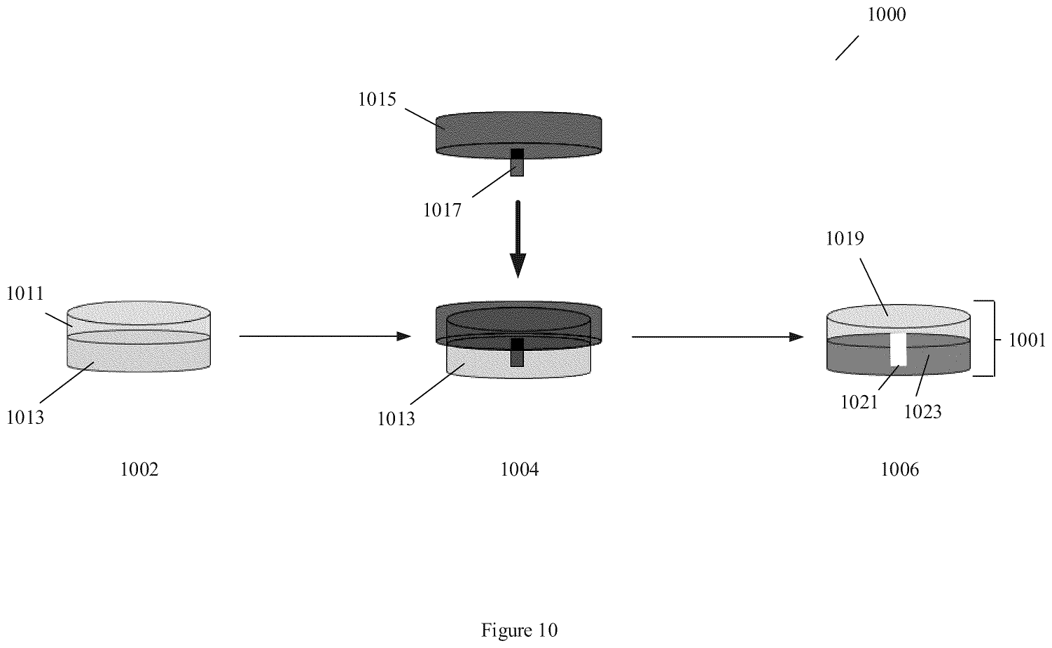

FIG. 10 illustrates a process 1000 using a mold to generate a sample receiver site 1021 according to various embodiments.

Step 1002 of the process 1000 may include depositing a solidifying solution 1013 into a structural support 1013 according to various embodiments. In various embodiments, the solidifying solution 1013 is in a liquid form at step 1002.

Step 1004 of the process 1000 may include inserting a mold 1015 having a protrusion 1017 into the solidifying solution 1013. In various embodiments, the solidifying solution 1013 is in a liquid form at step 1004.

Step 1006 of the process 1000 illustrates the completed sample receiver 1001 after the mold 1015 has been removed. Between steps 1004 and 1006 the solidifying agent 1013 is allowed to solidify into a scaffold 1023. In various embodiments, step 1006 includes removal of the protrusion 1017 from the scaffold 1023 to generate a sample receiving site.

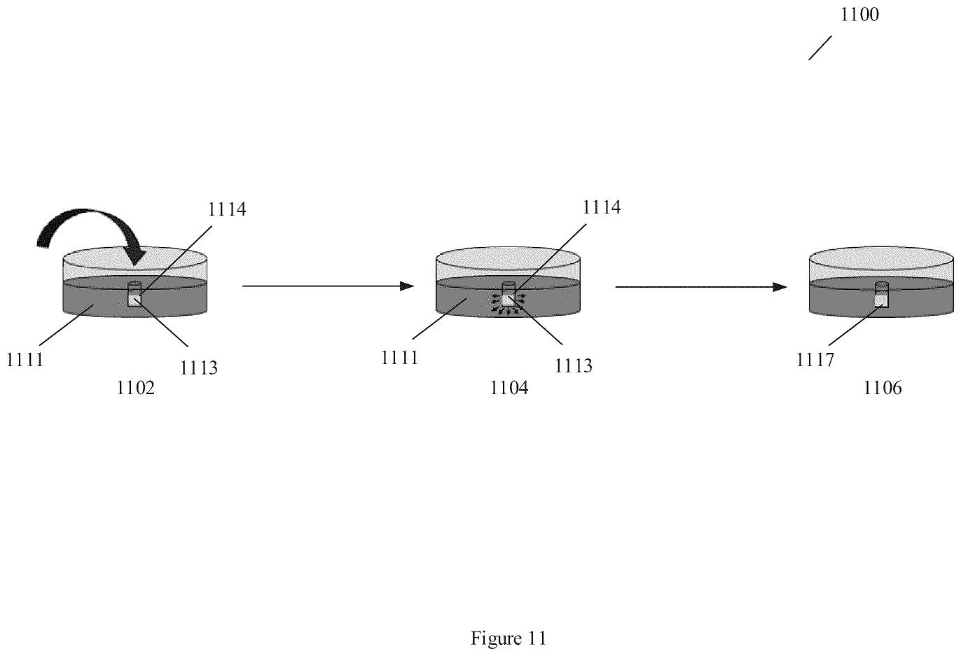

FIG. 11 illustrates a process for generating a depot from a long-acting injectable formulation according to various embodiments.

Step 1102 of the process 1100 comprises depositing a long-acting injectable formulation 1113 into a sample receiving site 1114. In various embodiments, the sample receiving site 1114 may be associated with a scaffold 1111. In various embodiments, the scaffold 1111 may take the form of any of the scaffolds described herein. In various embodiments, a long-acting injectable formulation may interact with the scaffold 1111 to form a depot.

In various embodiments, step 1101 comprises depositing an aqueous solution-based formulation (e.g., a drug solubilized by excipient) into the sample receiving site 1113. In various embodiments, step 1101 comprises depositing an aqueous suspension-based formulation (e.g., a drug suspension stabilized by excipient) into the sample receiving site 1113.

Step 1104 of the process 1100 comprises the step of transforming the combined formulation 1113 and scaffold 1111 into a depot 1117. A variety of different processes may occur depending on the formulation type. In most embodiments, a physical or chemical reaction may occur between the long-acting injectable drug formulation 1113 and the scaffold 1111 which results in a depot formation.

In embodiments where the formulation comprises an aqueous solution a solubilizing excipient may diffuse out of the long-acting injectable formulation and cause a drug compound of the formulation to precipitate and form a solid depot.

In embodiments where the formulation comprises an aqueous suspension, a stabilizing excipient may diffuse out of the long-acting injectable formulation and cause aggregation of suspended drug particles of the formulation to form a solid depot.

In embodiments where the formulation comprises an oil-based solution, a liquid depot is formed as the oil may not diffuse out of the long-acting injectable formulation.

In embodiments where the formulation comprises an oil-based suspension liquid/solid depot as the suspended drug particles aggregate and some may separate from the oil.

In embodiments where the formulation comprises microspheres, aggregate of microsphere may be used to form a solid depot.

In embodiments where the formulation comprises in situ forming implants, solid depot formation as phase separation or self-assembly of liquid crystals or sol-to-gel conversion of long-acting injectable formulations may occur.

Step 1106 of the process 1100 illustrates a formed depot 1117 after completion of the transformation step.

### IV. Example of a Biopredictive In Vitro Release Assay for Long-Acting Injectable Formulations

We developed technology for predicting a patient drug release profile for a long-acting injectable drug formulation allowing for substantially shorter development timelines. The in vitro biopredictive assay described herein and, in this example, may enable up to 250-day predictions in as little as five days of testing.

The feasibility studies for the systems and methods described herein were completed using a long-acting injectable formulation that included Lenacapavir (e.g. FA, FB, FC, and FD). Lenacapavir is a human deficiency virus-1 (HIV-1) capsid inhibitor which is currently approved as twice-yearly treatment for heavily treatment experienced people with human deficiency virus (HIV).

When Lenacapavir is injected into either a subcutaneous tissue or an intramuscular space, it precipitates to form a depot and releases the drug over an extended duration. Here, we have developed a system and method suitable for testing a variety of long-acting injectable formulations.

For long-acting drug injectable drug formulations, the lead time between clinical dosing and receiving meaningful data for evaluation of formulation impact is much longer than conventional injectables. As such, we needed a biopredictive assay to further develop Lenacapavir long-acting injectable formulation. We note that the biopredictive assay described herein has broad applicability well beyond testing of formulations for Lenacapavir. The biopredictive in vitro release test system and method described herein expedited and aided formulation selection to obtain the desired release and will be used for development of other long-acting injectable drug formulations. The system and method were tested using both solution and suspension based long-acting injectable formulations.

Additionally, formulations FE and FF described in more detail herein, include a non-Lenacapavir long-acting injectable formulation.

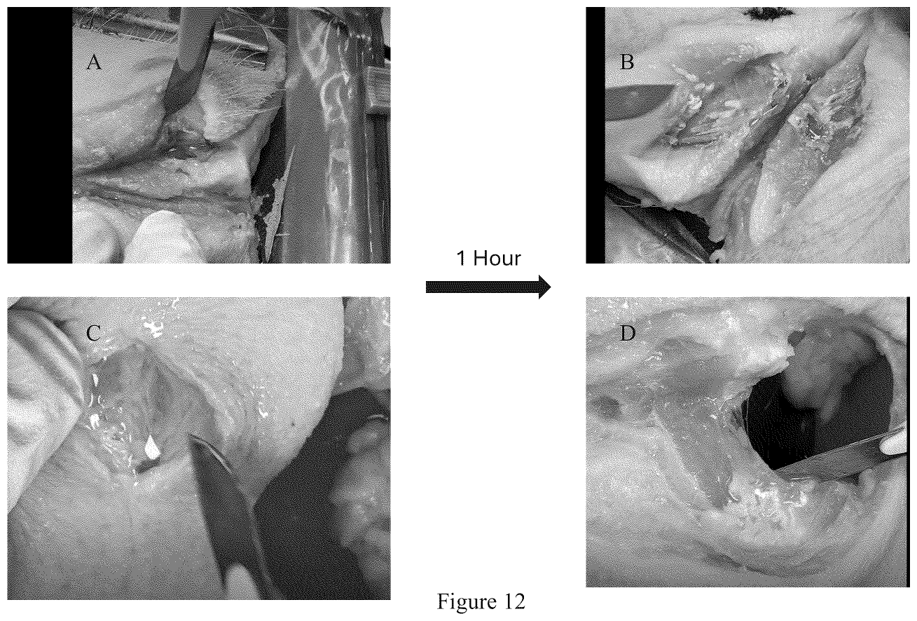

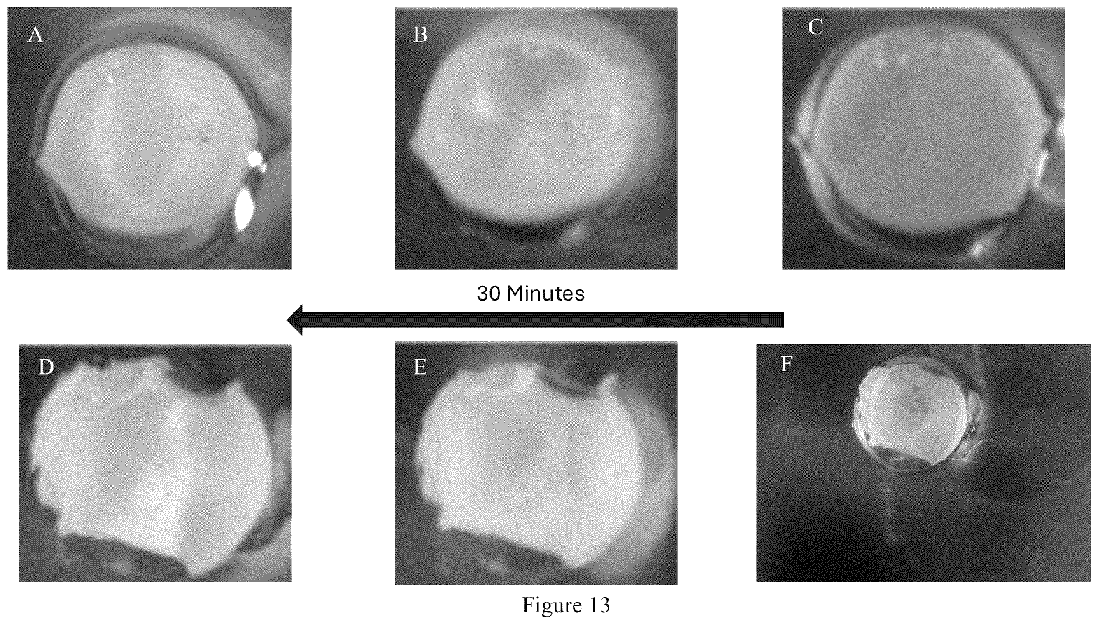

FIG. 12 photographically illustrates in vivo pig skin depot formation for comparison to in vitro agarose hydrogel depot formations shown in FIG. 13. FIG. 12, panels A and C show ex vivo pig skin prior to depot formation and FIG. 12, panels B and D show depot formation occurring after one hour. Subcutaneous deposition was completed on the samples. Panel A shows FA formulation deposition at time 0 and panel B shows the same sample after one hour has elapsed. Formation of a depot can be easily observed. Panel C shows FB formulation deposition at time 0 and panel D shows the same sample after one hour has elapsed.

FIG. 13 photographically illustrates in vitro agarose hydrogel well formation for comparison to ex vivo pig skin depot formations shown in FIG. 12. The scaffolds were formed from agar according to the methods described herein. Long-acting injectable formulations FA and FB were deposited into wells at time zero and observed for 30 minutes. FIG. 13, panels A, B, and C show in vitro depot formation, partially precipitated over 30 minutes. FIG. 13, panels D, E, and F show in vitro depot formation, completed precipitated over 30 minutes.

FIGS. 12 and 13 illustrate the similarity in depot formation of two long-acting injectable solution formulations in ex vivo pig skin and in vitro agarose hydrogel well. Sodium salt formulation precipitates partially both ex vivo and in vitro whereas free acid formulation precipitates completely within the same time duration. In various embodiments, ex vivo pig skin models were used to compare in vitro sample introductions. In various embodiments, depot formation was examined in ex vivo pig and compared to in vitro deposition formation in agar wells (e.g., volume at 5 ul). FIG. 13 shows visual representations of partially precipitated NaS depot formation and fully precipitated FA depot.

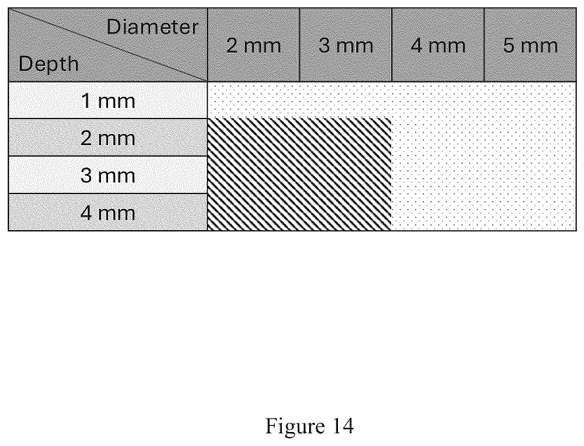

FIG. 14 schematically illustrates data showing optimized physical parameters of a depression or well for receiving a long-acting injectable formulation, according to various embodiments. The suitability of depression dimensions was tested by observing whether a long-acting injectable formulation leaked out of the depression and/or whether a precipitate was formed within a scaffold. We observed that a depression depth of 1 mm or less resulted in leakage of long-acting injectable formulation into a surrounding medium. Depths of 2 mm and more were observed to function optimally (e.g., reduced formulation leakage into surrounding medium combined with observable participate formulation between the formulation and scaffold). We also tested various diameters. We observed that depressions having diameters of 2 mm or 3 mm displayed minimized formulation leakage from the depression as well as formulation of a precipitate. As described herein, we also successfully tested use multiple depressions running parallel experiments.

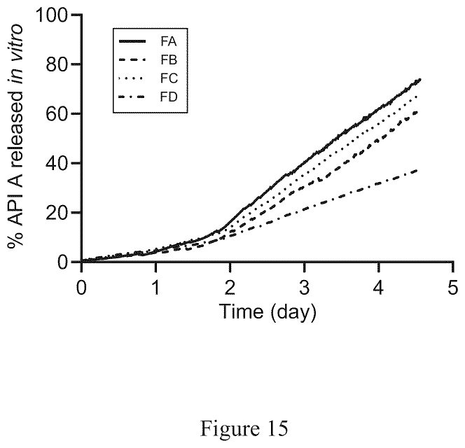

FIG. 15 is experimental data showing in vitro release profiles of four solution-based subcutaneous long-acting injectable formulations. The y-axis shows percentage of active pharmaceutical ingredient (API A) released over time (x-axis). Here, we tested four formulations over the course of five days. FA formulation includes low concentration of sodium salt form of API A. FB formulation includes low concentration of free acid form of API A. FC includes high concentration of sodium salt form of API A. FD includes high concentration of sodium salt form of API A with poloxamer excipient. Data illustrated in FIG. 14 is also shown in Table 1.

TABLE 1
In Vitro Based Long-Acting Injectable Mean Release Profiles

| Time (day) | FA (%) | FB (%) | FC (%) | FD (%) |
|---|---|---|---|---|
| 0.00 | 0.13 | 0.22 | 0.00 | 0.05 |
| 0.50 | 2.26 | 2.12 | 2.84 | 4.00 |
| 1.00 | 4.19 | 3.84 | 5.30 | 4.71 |
| 1.50 | 8.28 | 5.62 | 8.98 | 6.96 |
| 2.00 | 16.01 | 11.83 | 13.85 | 10.48 |
| 2.50 | 28.87 | 20.56 | 24.22 | 15.81 |
| 3.00 | 40.44 | 30.46 | 35.52 | 21.38 |
| 3.50 | 51.71 | 38.71 | 45.78 | 26.64 |
| 4.00 | 62.04 | 49.64 | 56.08 | 31.83 |
| 4.50 | 73.23 | 60.57 | 66.75 | 36.84 |
| 4.57 | 73.72 | 62.13 | 67.83 | 37.79 |

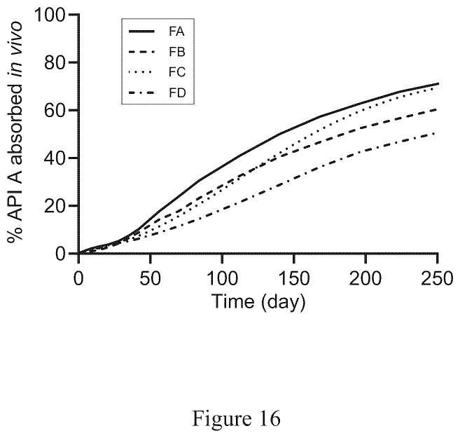

FIG. 16 is experimental data showing in vivo absorption profiles of four solution-based subcutaneous long-acting injectable formulations. The y-axis shows percentage of active pharmaceutical ingredient (API A) released over time (x-axis). Here, we tested four formulations over the course of 250 days. FA formulation includes low concentration of sodium salt form of API A. FB formulation includes low concentration of free acid form of API A. FC includes high concentration of sodium salt form of API A. FD includes high concentration of sodium salt form of API A with poloxamer excipient. Data illustrated in FIG. 15 is shown in Table 2.

TABLE 2
In Vivo Based Long-Acting Injectable Absorption Profiles

| Time (day) | FA (%) | FB (%) | FC (%) | FD (%) |
|---|---|---|---|---|
| 0.00 | 0 | 0.00 | 0.25 | 0.00 |
| 0.08 | 0.01 | 0.00 | 0.31 | 0.00 |
| 0.17 | 0.03 | 0.00 | 0.34 | 0.00 |
| 0.33 | 0.09 | 0.00 | 0.39 | 0.05 |
| 0.50 | 0.16 | 0.01 | 0.43 | 0.09 |
| 1.00 | 0.36 | 0.01 | 0.53 | 0.22 |
| 1.50 | 0.55 | 0.04 | 0.62 | 0.33 |
| 2.00 | 0.69 | 0.07 | 0.69 | 0.43 |
| 3.00 | 0.91 | 0.17 | 0.83 | 0.60 |
| 5.00 | 1.36 | 0.42 | 1.08 | 0.91 |
| 7.00 | 1.82 | 0.68 | 1.31 | 1.26 |
| 9.00 | 2.23 | 0.93 | 1.56 | 1.57 |
| 14.00 | 2.95 | 1.51 | 2.19 | 2.34 |
| 21.00 | 3.89 | 2.71 | 3.22 | 3.51 |
| 28.00 | 5.26 | 4.32 | 4.45 | 4.42 |
| 35.00 | 7.58 | 6.55 | 5.87 | 5.25 |
| 42.00 | 10.3 | 8.98 | 7.50 | 6.32 |
| 56.00 | 17.6 | 14.23 | 11.30 | 8.83 |
| 70.00 | 24.00 | 17.89 | 15.75 | 11.56 |
| 84.00 | 30.50 | 23.18 | 20.70 | 14.55 |
| 112 | 40.90 | 32.42 | 31.39 | 21.49 |
| 140 | 50.00 | 40.57 | 42.10 | 28.79 |
| 168 | 57.30 | 46.64 | 52.00 | 36.19 |
| 196 | 62.80 | 52.47 | 59.56 | 42.57 |
| 224 | 67.80 | 56.74 | 65.48 | 46.86 |
| 252 | 71.30 | 60.67 | — | 50.89 |

In predicting plasma concentration time profiles, we used two steps. First, we time scaled in vitro release profiles to use as our reference. Second, we obtained convolution of time-scaled in vitro release profile with unit input response (UIR) we obtained using intravenous bolus dose profile in a physiologically based pharmacokinetic model (PBPK).

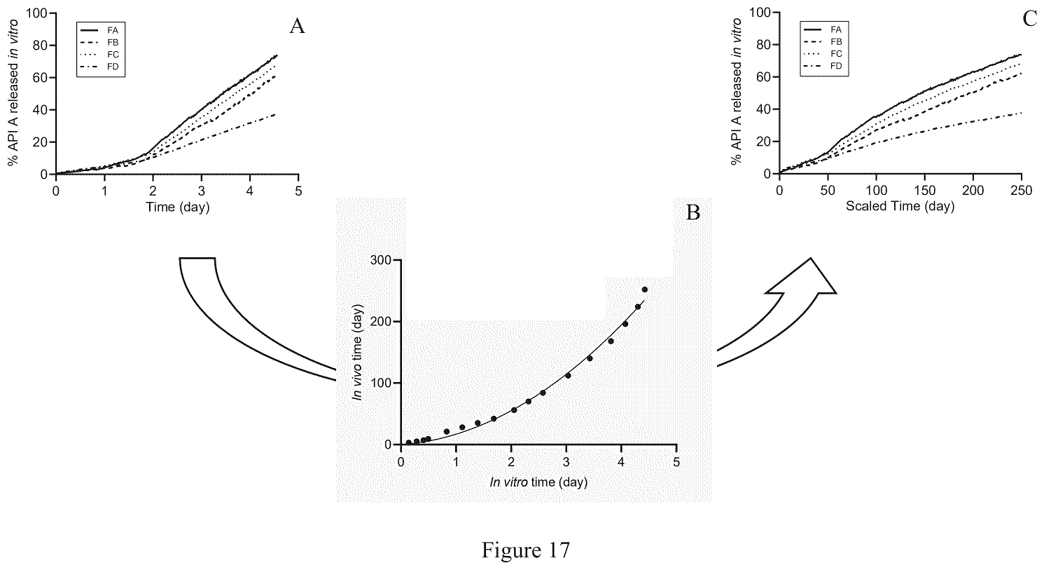

FIG. 17 is real data that was collected over five days using our in vitro assay (panel A) and was then processed using historically collected data (panel B) to generate predictive data that has been time-scaled from five days to 250 days (panel C). The data in panel A is shown in Table 1 and includes percentage API A released into the surrounding medium on the y-axis and time in days on the x-axis. Here, the development and utilization of a Levy plot (see panel B) was used to time scale the accelerated in vitro release profile of low concentration sodium salt long-acting injectable formulation FA obtained using the agarose gel well method over a period of 5 days to real time release occurring for 250 days. A Levy plot was used to depict the relationship between the corresponding in vitro and in vivo times for the same percent of active released/absorbed (in vivo days on y-axis, in vitro days on x-axis). In this case, a non-linear or quadratic relationship was established and was used to time scale accelerated in vitro release profile of sodium salt formulation. The quadric relationship shown in panel B can be represented by the formula y=10.56x²+6.509x.

Panel B and Table 3 include the necessary information for scaling in vitro release assay data to predictive time scaled data (see panel C and Table 4). As seen, the time scaled data shown in FIG. 17 matches the in vivo data seen in FIG. 16 very closely. The y-axis shows API A release into medium and the x-axis shows scaled time after application of the Levy plot.

TABLE 3
In Vitro to In Vivo Conversion Model

| In Vitro Time (Days) | In Vivo Time (Days) |
|---|---|
| 0.14 | 3 |
| 0.28 | 5 |
| 0.49 | 9 |
| 0.83 | 21 |
| 1.11 | 28 |
| 1.39 | 35 |
| 1.68 | 42 |
| 2.05 | 56 |
| 2.31 | 70 |
| 2.58 | 84 |
| 3.03 | 112 |
| 3.43 | 140 |
| 3.81 | 168 |
| 4.07 | 196 |
| 4.30 | 224 |
| 4.43 | 252 |

TABLE 4
Time Scaled In Vitro Experimental Data

| Time (days) | FA (%) | FB (%) | FC (%) | FD (%) |
|---|---|---|---|---|
| 0.00 | 0.13 | 0.22 | 0.00 | 0.05 |
| 0.05 | 0.25 | 0.36 | 0.08 | 0.15 |
| 0.10 | 0.34 | 0.47 | 0.13 | 0.26 |
| 0.50 | 0.59 | 0.77 | 0.69 | 1.00 |
| 1.00 | 0.83 | 0.94 | 1.20 | 1.38 |
| 10.06 | 3.16 | 2.82 | 3.70 | 4.01 |
| 50.20 | 13.51 | 9.96 | 12.32 | 9.29 |
| 100.32 | 35.59 | 27.20 | 30.91 | 19.24 |
| 150.27 | 51.31 | 38.60 | 45.04 | 26.39 |
| 200.29 | 63.45 | 50.24 | 57.18 | 32.49 |
| 250.09 | 73.72 | 62.13 | 67.83 | 37.79 |

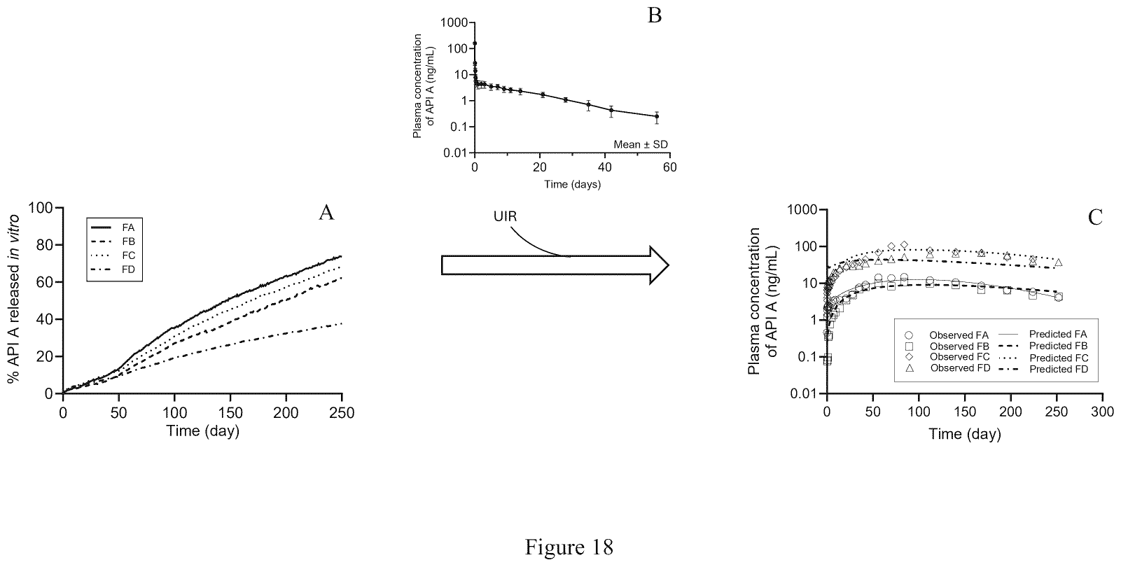

FIG. 18 illustrates use of convolution of time-scaled in vitro release profile data (panel A, Table 4) with unit input response (UIR) obtained using an intravenous bolus dose (panel B, Table 5) to generate predictive plasma concentration-time profiles (panel C, Table 6). Panel A includes time-scaled mean in vitro release profiles of various solution-based subcutaneous long acting injectables of API. Panel C includes observed versus predicted mean plasma concentration-time profiles in humans for various solution-based subcutaneous long acting injectables of API A.

TABLE 5
Unit Input Response Convolution

| Time (Days) | Plasma Concentration of API A (ng/mL) |
|---|---|
| 0.00 | 0.00 |
| 0.04 | 161.5 |
| 0.08 | 27.71 |
| 0.17 | 14.00 |
| 0.25 | 7.73 |
| 0.33 | 5.71 |
| 0.50 | 4.68 |
| 1.00 | 4.33 |
| 2.00 | 4.37 |
| 3.00 | 4.29 |
| 5.00 | 3.49 |
| 7.00 | 3.45 |
| 9.00 | 2.84 |
| 11.00 | 2.61 |
| 14.00 | 2.33 |
| 20.92 | 1.71 |
| 27.92 | 1.10 |
| 34.96 | 0.71 |
| 41.96 | 0.43 |
| 55.96 | 0.25 |

TABLE 6A
Observed and Predicted Plasma Concentration Profiles (FA)

| Time (Days) | Observed (%) | Predicted (%) |
|---|---|---|
| 0.00 | 0.00 | 0.00 |
| 0.08 | 0.46 | 3.86 |
| 0.17 | 1.23 | 3.85 |
| 0.33 | 1.92 | 3.34 |
| 0.50 | 2.22 | 2.96 |
| 1.00 | 2.33 | 2.57 |
| 1.50 | 2.43 | 2.54 |
| 2.00 | 2.09 | 2.59 |
| 3.00 | 2.30 | 2.77 |
| 5.00 | 2.89 | 3.19 |
| 7.00 | 3.30 | 3.60 |
| 9.00 | 3.56 | 3.99 |
| 14.00 | 3.47 | 4.90 |
| 21.00 | 3.90 | 6.07 |
| 28.00 | 5.01 | 7.16 |
| 35.00 | 7.57 | 8.16 |
| 42.00 | 9.06 | 9.08 |
| 56.00 | 14.30 | 10.61 |
| 70.00 | 13.80 | 11.72 |
| 84.00 | 14.75 | 12.39 |
| 112.00 | 12.04 | 15.52 |
| 140.00 | 10.61 | 11.43 |
| 168.00 | 8.60 | 9.63 |
| 196.00 | 6.53 | 7.61 |
| 224.00 | 5.89 | 5.70 |
| 252.00 | 4.09 | — |

TABLE 6B
Observed and Predicted Plasma Concentration Profiles (FB)

| Time (Days) | Observed (%) | Predicted (%) |
|---|---|---|
| 0.00 | 0.00 | 0.00 |
| 0.08 | 0.00 | 0.11 |
| 0.17 | 0.00 | 0.17 |
| 0.33 | 0.08 | 0.27 |
| 0.50 | 0.08 | 0.33 |
| 1.00 | 0.10 | 0.46 |
| 1.50 | 0.35 | 0.57 |
| 2.00 | 0.39 | 0.67 |
| 3.00 | 0.77 | 0.87 |
| 5.00 | 1.15 | 1.27 |
| 7.00 | 1.39 | 1.67 |
| 9.00 | 1.64 | 2.05 |
| 14.00 | 2.08 | 2.97 |
| 21.00 | 3.49 | 4.12 |
| 28.00 | 4.74 | 5.11 |
| 35.00 | 6.84 | 5.94 |
| 42.00 | 7.93 | 6.64 |
| 56.00 | 10.13 | 7.69 |
| 70.00 | 8.29 | 8.38 |
| 84.00 | 10.89 | 8.80 |
| 112.00 | 9.84 | 9.06 |
| 140.00 | 8.97 | 8.80 |
| 168.00 | 6.71 | 8.22 |
| 196.00 | 6.49 | 7.48 |
| 224.00 | 4.71 | 6.68 |
| 252.00 | 4.38 | — |

TABLE 6C
Observed and Predicted Plasma Concentration Profiles (FC)

| Time (Days) | Observed (%) | Predicted (%) | |
|---|---|---|---|
| 0.00 | 1.40 | 0.00 | |
| 0.08 | 2.07 | 1.01 | |
| 0.17 | 3.45 | 1.74 | |
| 0.33 | 5.29 | 2.75 | |
| 0.50 | 6.27 | 3.44 | |
| 1.00 | 7.27 | 4.90 | |
| 1.50 | 7.23 | 6.11 | |
| 2.00 | 7.43 | 7.26 | |
| 3.00 | 8.71 | 9.46 | |
| 5.00 | 10.76 | 13.74 | |
| 7.00 | 13.66 | 17.90 | |
| 9.00 | 15.13 | 21.93 | |
| 14.00 | 22.18 | 31.34 | |
| 21.00 | 27.20 | 42.71 | |
| 28.00 | 32.91 | 52.03 | |
| 35.00 | 37.64 | 59.53 | |
| 42.00 | 44.91 | 65.50 | |
| 56.00 | 68.20 | 73.82 | |
| 70.00 | 102.00 | 78.63 | |
| 84.00 | 112.82 | 81.01 | |
| 112.00 | 77.95 | 81.01 | |
| 140.00 | 70.88 | 76.97 | |
| 168.00 | 67.28 | 71.19 | (165 days) |
| 196.00 | 50.23 | 62.58 | (195 days) |
| 224.00 | 45.50 | 53.04 | (225 days) |
| 252.00 | — | 250 44.98 | (250 days) |

TABLE 6D
Observed and Predicted Plasma Concentration Profiles (FD)

| Time (Days) | Observed (%) | Predicted (%) | |
|---|---|---|---|
| 0.00 | 0.00 | 0.00 | |
| 0.08 | 2.77 | — | |
| 0.17 | 4.50 | — | |
| 0.33 | 8.16 | — | |
| 0.50 | 9.74 | — | |
| 1.00 | 11.97 | 28.29 | |
| 1.50 | 11.42 | 26.83 | |
| 2.00 | 11.58 | 26.63 | |
| 3.00 | 12.385 | 27.18 | |
| 5.00 | 15.05 | 29.00 | |
| 7.00 | 19.22 | 30.76 | |
| 9.00 | 19.51 | 32.34 | |
| 14.00 | 25.45 | 35.51 | |
| 21.00 | 30.84 | 38.64 | |
| 28.00 | 29.65 | 40.74 | |
| 35.00 | 30.08 | 42.14 | |
| 42.00 | 34.48 | 43.04 | |
| 56.00 | 41.11 | 43.82 | |
| 70.00 | 45.99 | 43.71 | |
| 84.00 | 51.20 | 43.06 | |
| 112.00 | 62.22 | 40.82 | |
| 140.00 | 65.49 | 37.95 | |
| 168.00 | 66.91 | 34.87 | (167.5 days) |
| 196.00 | 57.76 | 31.74 | (195 days) |
| 224.00 | 39.52 | 28.67 | (222.5 days) |
| 252.00 | 37.38 | 25.77 | (250 days) |

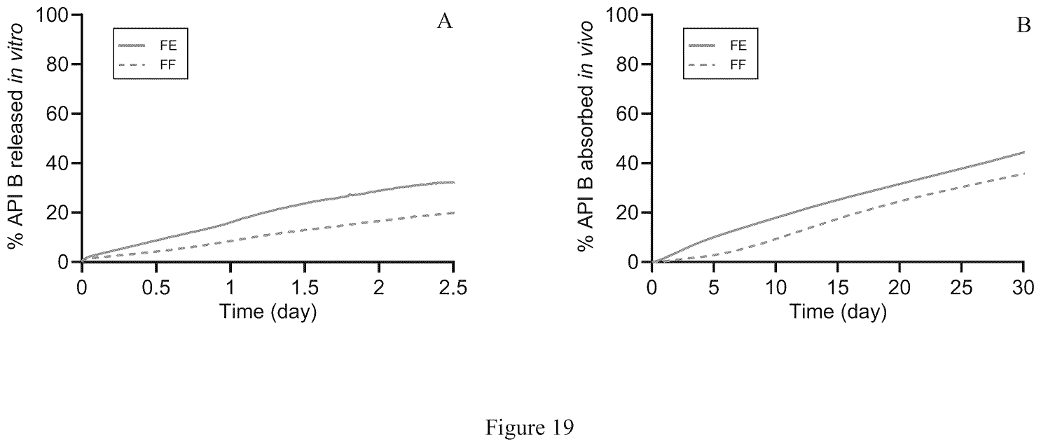

FIG. 19, panel A is experimental data showing mean in vitro release profiles of subcutaneous solution versus suspension long acting injectables of API B. FIG. 19, panel B is experimental data showing mean deconvoluted in vivo absorption profiles of subcutaneous solution versus suspension long acting injectables of API B. The FE plot is solution-based LAI formulation of API B. The FF plot is solution-based LAI formulation of API B. Table 7 and 8 include values depicted in FIG. 19.

TABLE 7
In Vitro Release Profiles

| Time (day) | FE (%) | FF (%) |
|---|---|---|
| 0 | 0.00 | 0.02 |
| 0.25 | 5.15 | 2.67 |
| 0.33 | 6.34 | 3.18 |
| 0.5 | 8.68 | 4.22 |
| 1 | 16.01 | 8.47 |
| 1.5 | 23.69 | 12.96 |
| 2.0 | 28.85 | 16.55 |
| 2.5 | 32.04 | 19.85 |

TABLE 8
In Vivo Release Profiles

| Time (day) | FE (%) | FF (%) |
|---|---|---|
| 0 | 0 | 0 |
| 0.04 | 0.01 | — |
| 0.08 | 0.03 | — |
| 0.17 | 0.09 | — |
| 0.25 | 0.18 | — |
| 0.33 | 0.29 | — |
| 0.5 | 0.54 | — |
| 1 | 1.51 | 0.33 |
| 2 | 3.82 | 0.9 |
| 3 | 6.13 | 1.5 |
| 4 | 8.18 | 2.08 |
| 5 | 10 | 2.75 |
| 6 | 11.7 | 3.73 |
| 7 | 13.23 | 4.85 |
| 9 | 16.38 | 7.65 |
| 13 | 22.35 | 14.23 |
| 16 | 26.43 | 19.08 |
| 20 | 31.55 | 24.55 |
| 23 | 35.28 | 28.1 |
| 27 | 40.28 | 32.48 |
| 30 | 44.28 | 35.65 |

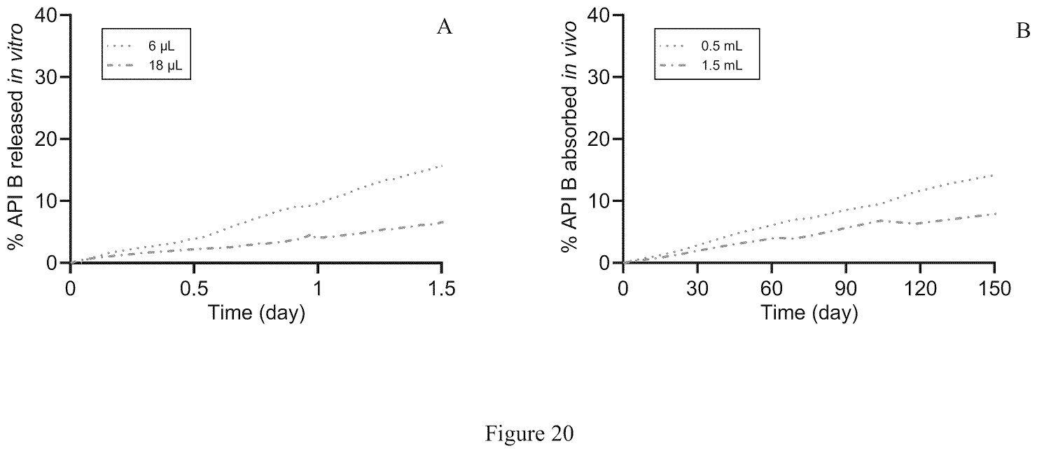

FIG. 20 is experimental data comparing low dose and high dose formulations in vitro (panel A) and in vivo (panel B). We observed that higher doses (i.e. increased dose volume with the same concentration) leads to slower percent release. Similar rank ordering was observed in both in vitro and in vivo studies. The FE plot is solution-based LAI formulation of API A. The FF plot is solution-based LAI formulation of API B. Data in FIG. 20, panel A is tabulated in Table 9. Data in FIG. 20, panel B is tabulated in Table 10.

TABLE 9
In Vitro Release Profiles

| Time (day) | FE (%) | FF (%) |
|---|---|---|
| 0 | 0 | 0 |
| 0.25 | 2.24 | 1.42 |
| 0.33 | 2.69 | 1.72 |
| 0.5 | 3.91 | 2.20 |
| 1 | 9.58 | 4.08 |
| 1.5 | 15.65 | 6.57 |

TABLE 10
In Vivo Release Profiles

| Time (day) | FE (%) | FF (%) |
|---|---|---|
| 0.25 | 0.03 | 0.02 |
| 0.33 | 0.04 | 0.03 |
| 0.5 | 0.06 | 0.04 |
| 1 | 0.11 | 0.08 |
| 2 | 0.22 | 0.15 |
| 3 | 0.32 | 0.22 |
| 4 | 0.40 | 0.28 |
| 5 | 0.48 | 0.33 |
| 6 | 0.55 | 0.38 |
| 7 | 0.63 | 0.43 |
| 9 | 0.77 | 0.52 |
| 13 | 1.07 | 0.73 |
| 16 | 1.32 | 0.91 |
| 20 | 1.71 | 1.17 |
| 23 | 2.03 | 1.39 |
| 27 | 2.48 | 1.71 |
| 34 | 3.32 | 2.25 |
| 41 | 4.16 | 2.75 |
| 48 | 4.50 | 3.20 |
| 55 | 5.59 | 3.63 |
| 62 | 6.27 | 4.04 |
| 69 | 6.98 | 3.91 |
| 76 | 7.25 | 4.42 |
| 90 | 8.57 | 5.66 |
| 104 | 9.53 | 6.80 |
| 118 | 11.46 | 6.31 |
| 132 | 12.83 | 7.02 |
| 160 | 14.93 | 8.39 |

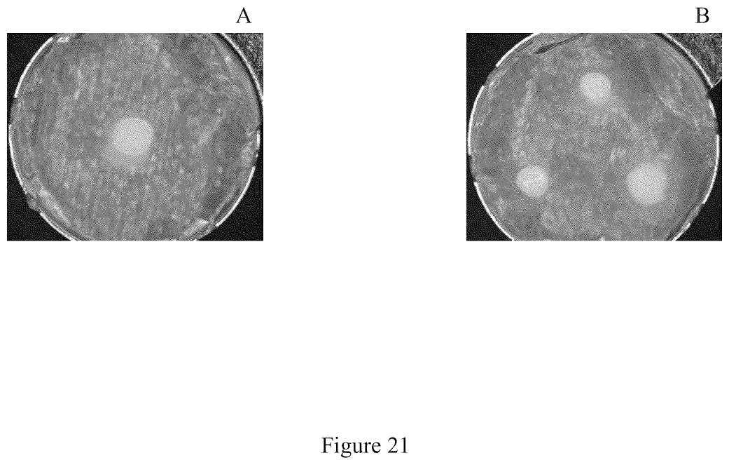

FIG. 21, panel A shows a depot configuration for the low dose in vitro assay and FIG. 20, panel B shows a depot configuration for the high dose in vitro assay of FIG. 20. Panel A shows a single well configuration for testing a 6 ul formulation. A 6 ul formulation simulates a 0.5 mL dose in vivo. Panel B shows a three-well configuration for testing an 18 ul formulation. An 18 ul formulation simulates a 0.5 mL dose in vivo.

### EQUIVALENCE

Those skilled in the art will recognize or be able to ascertain using no more than routine experimentation, many equivalents to the specified embodiments of the technologies described herein. It is to be understood that the technologies encompass all variants, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the technologies can be explicitly excluded from the claims, regardless of whether the specific exclusion is recited in the specification.

### RECITATION OF EMBODIMENTS

Embodiment 1: A system for predicting a drug release profile of a long-acting injectable formulation comprising a chamber for receiving a fluid, a sample receiver disposed within the chamber, and a mixing system for mixing the fluid within the chamber. The sample receiver comprises a structural support for supporting a scaffold and a sample receiving site associated with the scaffold.

Embodiment 2: The system for predicting a drug release profile of a long-acting injectable formulation of embodiment 1, wherein the sample receiving site is located within an interior of the scaffold.

Embodiment 3: The system for predicting a drug release profile of a long-acting injectable formulation of embodiment 1, wherein the sample receiving site comprises a surface of the scaffold.

Embodiment 4: The system for predicting a drug release profile of a long-acting injectable formulation of embodiment 1, wherein the sample receiving site comprises a depression associated with the scaffold.

Embodiment 5: The system for predicting a drug release profile of a long-acting injectable formulation of embodiment 4, wherein the depression comprises a sidewall extending into a surface to a floor, wherein the sidewall and floor form the depression.

Embodiment 6: The system for predicting a drug release profile of a long-acting injectable formulation of any one of embodiments 4-5, wherein the depression is conical.

Embodiment 7: The system for predicting a drug release profile of a long-acting injectable formulation of any one of embodiments 4-5, wherein the depression is cylindrical.

Embodiment 8: The system for predicting a drug release profile of a long-acting injectable formulation of embodiment 7, wherein the depression comprises a depth ranging between 2 mm and about 4 mm.

Embodiment 9: The system for predicting a drug release profile of a long-acting injectable formulation of embodiment 7, wherein the depression comprises a depth of 3 mm.

Embodiment 10: The system for predicting a drug release profile of a long-acting injectable formulation of embodiment 7, wherein the depression comprises a diameter ranging between 2 mm and 3 mm.

Embodiment 11: The system for predicting a drug release profile of a long-acting injectable formulation of embodiment 7, wherein the depression comprises a diameter of 2.5 mm.

Embodiment 12: The system for predicting a drug release profile of a long-acting injectable formulation according to any one of the preceding embodiments, wherein the scaffold comprises a hydrogel.

Embodiment 13: The system for predicting a drug release profile of a long-acting injectable formulation of embodiment 12, wherein the hydrogel comprises between about 0.5% to 2% agarose.

Embodiment 14: The system for predicting a drug release profile of a long-acting injectable formulation according to any one of embodiments 12-13, wherein the hydrogel comprises collagen.

Embodiment 15: The system for predicting a drug release profile of a long-acting injectable formulation according to any one of embodiments 12-14, wherein the hydrogel comprises polyacrylamide.

Embodiment 16: The system for predicting a drug release profile of a long-acting injectable formulation according to any one of embodiments 12-15, wherein the hydrogel comprises a phosphate buffered saline at pH 7.4.

Embodiment 17: The system for predicting a drug release profile of a long-acting injectable formulation according to any one of the preceding embodiments, wherein the scaffold comprises albumin.

Embodiment 18: The system for predicting a drug release profile of a long-acting injectable formulation according to any one of the preceding embodiments, wherein the scaffold comprises at least one enzyme.

Embodiment 19: The system for predicting a drug release profile of a long-acting injectable formulation of embodiment 18, wherein the at least one enzyme includes an esterase, a phosphatase, an amidase, or a hydrolase.

Embodiment 20: The system for predicting a drug release profile of a long-acting injectable formulation according to any one of the preceding embodiments, wherein the chamber comprises a sample port.

Embodiment 21: The system for predicting a drug release profile of a long-acting injectable formulation of embodiment 20, wherein the sample port is fluidically connected to a fluid collection apparatus.

Embodiment 22: The system for predicting a drug release profile of a long-acting injectable formulation according to any one of the embodiments, further comprising a lid for sealing the chamber at an opening.

Embodiment 23: The system for predicting a drug release profile of a long-acting injectable formulation of embodiment 22, further comprising a probe extending through the lid, wherein the probe is at least partially disposed within the chamber.

Embodiment 24: The system for predicting a drug release profile of a long-acting injectable formulation of embodiment 23, wherein the probe comprises a fiber optic ultraviolet (UV) probe.

Embodiment 25: The system for predicting a drug release profile of a long-acting injectable formulation according to any one of the preceding embodiments, wherein the chamber further comprises an inlet for transferring the fluid into the chamber and an outlet for transferring the fluid out of the chamber.

Embodiment 26: The system for predicting a drug release profile of a long-acting injectable formulation of embodiment 25, further comprising a bead situated adjacent to the outlet.

Embodiment 27: The system for predicting a drug release profile of a long-acting injectable formulation according to any one of embodiments 25-26, wherein the sample receiver is positioned between the inlet and the outlet, and wherein a space within the chamber and between the sample receiver and the outlet is at least partially filled with a solid matrix to support the sample receiver.

Embodiment 28: The system for predicting a drug release profile of a long-acting injectable formulation according to any one of embodiments 25-27, further comprising a fluid sample collection apparatus fluidically connected to the outlet.

Embodiment 29: The system for predicting a drug release profile of a long-acting injectable formulation of embodiment 28, wherein the fluid sample collection apparatus is detachable from a fluidic connection for off-line analysis of the fluid.

Embodiment 30: The system for predicting a drug release profile of a long-acting injectable formulation according to any one of embodiments 27-29, further comprising a probe positioned at or near a fluidic connection.

Embodiment 31: The system for predicting a drug release profile of a long-acting injectable formulation of embodiment 30, wherein the fluidic connection comprises a tube.

Embodiment 32: The system for predicting a drug release profile of a long-acting injectable formulation according to any one of embodiments 30-31, wherein the probe measures a quality of the fluid.

Embodiment 33: The system for predicting a drug release profile of a long-acting injectable formulation of embodiment 32, wherein the measurement includes opacity.

Embodiment 34: The system for predicting a drug release profile of a long-acting injectable formulation of embodiment 32, wherein the measurement includes pH.

Embodiment 35: The system for predicting a drug release profile of a long-acting injectable formulation of embodiment 32, wherein the measurement includes fluorescence.

Embodiment 36: The system for predicting a drug release profile of a long-acting injectable formulation of embodiment 32, wherein the measurement includes ultraviolet light.

Embodiment 37: The system for predicting a drug release profile of a long-acting injectable formulation according to any one of the preceding embodiments, wherein the mixing system comprises a mixing element disposed within the chamber.

Embodiment 38: The system for predicting a drug release profile of a long-acting injectable formulation of embodiment 37, wherein the mixing element comprises a paddle.

Embodiment 39: The system for predicting a drug release profile of a long-acting injectable formulation of embodiment 37, wherein the mixing element comprises a stir bar.

Embodiment 40: The system for predicting a drug release profile of a long-acting injectable formulation according to any one of the preceding embodiments, wherein the structural support includes a floor having at least one upstanding sidewall for supporting the scaffold.

Embodiment 41: The system for predicting a drug release profile of a long-acting injectable formulation of embodiment 40, wherein the structural support comprises a container, a receptacle, a vessel, a canister, a drum, a cassette, a well, a cup, a box, or a receptible having at least one open end.

Embodiment 42: The system for predicting a drug release profile of a long-acting injectable formulation according to any one of the preceding embodiments, wherein the fluid includes a biorelevant media.

Embodiment 43: The system for predicting a drug release profile of a long-acting injectable formulation of embodiment 42, wherein the biorelevant media comprises between 2 to 4 percent bovine serum albumin (BSA) or human serum albumin (HAS).

Embodiment 44: The system for predicting a drug release profile of a long-acting injectable formulation of embodiment 43, wherein the fluid further includes 0.02% sodium azide.

Embodiment 45: A method for predicting a drug release profile of a long-acting injectable formulation, comprising generating a depot by combining an injection formulation with a scaffold, wherein the injection formulation comprises a drug compound and an excipient, contacting the depot with a fluid, wherein a compound diffuses from the depot to the fluid, and analyzing the fluid at a plurality of timepoints.

Embodiment 46: The method for predicting a drug release profile of a long-acting injectable formulation of embodiment 45, wherein the step of analyzing comprises recording at least two measurements of a characteristic of the fluid at different times, generating an in vitro drug release profile using the at least two measurements, and predicting an in vivo drug release profile for the injection formulation by comparing the in vitro drug release profile to a reference.

Embodiment 47: The method for predicting a drug release profile for a long-acting injectable formulation of embodiment 46, wherein the characteristic is influenced by a quantity of the compound in the fluid.

Embodiment 48: The method for predicting a drug release profile for a long-acting injectable formulation according to any one of embodiments 45-47, wherein the compound comprises an analyte that is detectable by a probe.

Embodiment 49: The method for predicting a drug release profile of a long-acting injectable formulation according to any one of embodiments 45-48, wherein the step of generating the in vitro release profile requires less than 30 days and the predicted drug release profile extends to at least 180 days.

Embodiment 50: The method for predicting a drug release profile of a long-acting injectable formulation according to any one of embodiments 45-48, wherein the step of generating the in vitro release profile requires five days or less and the predicted drug release profile extends to at least 50 days.

Embodiment 51: The method for predicting a drug release profile of a long-acting injectable formulation according to any one of embodiments 45-48, wherein the step of generating the in vitro release profile requires five days or less and the predicted drug release profile extends to at least 100 days.

Embodiment 52: The method for predicting a drug release profile of a long-acting injectable formulation according to any one of embodiments 45-48, wherein the step of generating the in vitro release profile requires five days or less and the predicted drug release profile extends to at least 150 days.

Embodiment 53: The method for predicting a drug release profile of a long-acting injectable formulation according to any one of embodiments 45-48, wherein the step of generating the in vitro release profile requires five days or less and the predicted drug release profile extends to at least 200 days.

Embodiment 54: The method for predicting a drug release profile of a long-acting injectable formulation according to any one of embodiments 45-48, wherein the step of generating the in vitro release profile requires five days or less and the predicted drug release profile extends to at least 250 days.

Embodiment 55: The method for predicting a drug release profile of a long-acting injectable formulation according to any one of embodiments 45-54, further comprising determining a concentration of the compound in the fluid.

Embodiment 56: The method for predicting a drug release profile of a long-acting injectable formulation according to any one of embodiments 45-55, wherein the step of generating the depot comprises initiating a fluid solidification process, injecting the drug formulation into the scaffold during the solidification process, and completing the fluid solidification process.

Embodiment 57: The method for predicting a drug release profile of a long-acting injectable formulation according to any one of embodiments 45-55, wherein the step of generating the depot comprises initiating a fluid solidification process, injecting the drug formulation into the scaffold after the solidification process, and completing the fluid solidification process.

Embodiment 58: The method for predicting a drug release profile of a long-acting injectable formulation of embodiment 57, wherein the injecting step uses an autoinjector.

Embodiment 59: The method for predicting a drug release profile of a long-acting injectable formulation of embodiment 57, wherein the injecting step uses a manual injection method.

Embodiment 60: The method for predicting a drug release profile of a long-acting injectable formulation according to any one of embodiments 45-59, wherein the scaffold includes a depression in a hydrogel and the step of generating a depot by combining a scaffold and a sample comprises depositing the sample into the depression.

Embodiment 61: The method for predicting a drug release profile of a long-acting injectable formulation of embodiment 60, wherein the hydrogel comprises a surface and a sidewall extending away from the surface and into the hydrogel to a floor forming the depression.

Embodiment 62: The method for predicting a drug release profile of a long-acting injectable formulation of embodiment 61, further comprising applying at least one mold to a solidifying solution, wherein the mold includes at least one protrusion extending into the solidifying solution, solidifying the solidifying solution into a hydrogel, and removing the mold to generate the depression.

Embodiment 63: The method for predicting a drug release profile of a long-acting injectable formulation of embodiment of embodiment 62, wherein the mold includes three protrusions.

Embodiment 64: The method for predicting a drug release profile of a long-acting injectable formulation according to any one of embodiments 62-63, wherein the protrusion is cylindrical.

Embodiment 65: The method for predicting a drug release profile of a long-acting injectable formulation according to any one of embodiments 62-64, wherein the solidifying solution comprises between 0.5% to 2% agarose and a phosphate buffered saline at pH 7.4.

Embodiment 66: The method for predicting a drug release profile of a long-acting injectable formulation according to any one of embodiments 45-65, wherein the step of generating the depot further comprises diffusing an excipient from the combined injection formulation and scaffold.

Embodiment 67: The method for predicting a drug release profile of a long-acting injectable formulation according to any one of embodiment 45-66, wherein the characteristic of the fluid includes a drug compound concentration.

Embodiment 68: The method for predicting a drug release profile of a long-acting injectable formulation according to any one of embodiments 45-67, wherein the characteristic of the fluid includes a drug analyte.

Embodiment 69: The method for predicting a drug release profile of a long-acting injectable formulation of embodiment 68, wherein the drug analyte includes a salt.

Embodiment 70: The method for predicting a drug release profile of a long-acting injectable formulation of embodiment 68, wherein the drug analyte includes a prodrug.

Embodiment 71: The method for predicting a drug release profile of a long-acting injectable formulation of embodiment 70, further comprising the step of cleaving the prodrug using an enzyme.

Embodiment 72: The method for predicting a drug release profile of a long-acting injectable formulation of embodiment 71, wherein the enzyme is associated with the fluid.

Embodiment 73: The method for predicting a drug release profile of a long-acting injectable formulation of embodiment 71, wherein the enzyme is associated with the scaffold.

Embodiment 74: The method for predicting a drug release profile of a long-acting injectable formulation of embodiment 68, wherein the drug analyte includes a reactionary product of the drug compound.

Embodiment 75: The method for predicting a drug release profile of a long-acting injectable formulation according to any one of embodiments 45-74, wherein the characteristic of the fluid includes an opacity.

Embodiment 70: The method for predicting a drug release profile of a long-acting injectable formulation according to any one of embodiments 45-74, wherein the characteristic of the fluid includes a pH.

Embodiment 77: The method for predicting a drug release profile of a long-acting injectable formulation according to any one of embodiments 45-74, further comprising the step of mixing the fluid.

Embodiment 78: The method for predicting a drug release profile of a long-acting injectable formulation of embodiment 77, wherein the mixing is continuous.

Embodiment 79: The method for predicting a drug release profile of a long-acting injectable formulation of embodiment 77, wherein the mixing is intermittent.

Embodiment 80: The method for predicting a drug release profile of a long-acting injectable formulation according to any one of embodiments 45-79, wherein the characteristic of the fluid includes a free acid.

Embodiment 81: The method for predicting a drug release profile of a long-acting injectable formulation according to any one of embodiments 45-79, wherein the characteristic of the fluid includes a free base.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The foregoing written specification is considered to be sufficient to enable one skilled in the art to practice the invention. The foregoing description and Examples that follow detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. A system for predicting a drug release profile of a long-acting injectable formulation, comprising:
a chamber for receiving a fluid;
a sample receiver disposed within the chamber, wherein the sample receiver comprises:
a structural support for supporting a scaffold; and
a sample receiving site associated with the scaffold; and
a mixing system for mixing the fluid within the chamber.

2. The system for predicting a drug release profile of a long-acting injectable formulation of claim 1, wherein the sample receiving site comprises a depression associated with the scaffold.

3. The system for predicting a drug release profile of a long-acting injectable formulation of claim 2, wherein the depression comprises:
a sidewall extending into a surface to a floor, wherein the sidewall and floor form the depression.

4. The system for predicting a drug release profile of a long-acting injectable formulation of claim 3, wherein the depression is cylindrical.

5. The system for predicting a drug release profile of a long-acting injectable formulation of claim 4, wherein the depression comprises a depth ranging between 2 mm and 4 mm.

6. The system for predicting a drug release profile of a long-acting injectable formulation of claim 4, wherein the depression comprises a depth of 3 mm.

7. The system for predicting a drug release profile of a long-acting injectable formulation of claim 4, wherein the depression comprises a diameter ranging between 2 mm and 3 mm.

8. The system for predicting a drug release profile of a long-acting injectable formulation of claim 4, wherein the depression comprises a diameter of 2.5 mm.

9. The system for predicting a drug release profile of a long-acting injectable formulation of claim 1, wherein the scaffold comprises a hydrogel.

10. The system for predicting a drug release profile of a long-acting injectable formulation of claim 9, wherein the hydrogel comprises between about 0.5% to 2% agarose.

11. The system for predicting a drug release profile of a long-acting injectable formulation of claim 10, wherein the hydrogel comprises
a phosphate buffered saline at pH 7.4.

12. The system for predicting a drug release profile of a long-acting injectable formulation of claim 1, wherein the scaffold comprises albumin.

13. The system for predicting a drug release profile of a long-acting injectable formulation of claim 1, wherein the scaffold comprises at least one enzyme.

14. The system for predicting a drug release profile of a long-acting injectable formulation of claim 13, wherein the at least one enzyme includes an esterase, a phosphatase, an amidase, or a hydrolase.

15. The system for predicting a drug release profile of a long-acting injectable formulation of claim 1, wherein the chamber comprises a sample port and the sample port is fluidically connected to a fluid collection apparatus.

16. (canceled)

17. The system for predicting a drug release profile of a long-acting injectable formulation of claim 1, further comprising:
a lid for sealing the chamber at an opening.

18. The system for predicting a drug release profile of a long-acting injectable formulation of claim 17, further comprising:
a probe extending through the lid, wherein the probe is at least partially disposed within the chamber.

19. The system for predicting a drug release profile of a long-acting injectable formulation of claim 1, wherein the chamber further comprises:
an inlet for transferring the fluid into the chamber; and
an outlet for transferring the fluid out of the chamber.

20. The system for predicting a drug release profile of a long-acting injectable formulation of claim 19, further comprising a bead situated adjacent to the outlet.

21. The system for predicting a drug release profile of a long-acting injectable formulation of claim 1, wherein the structural support includes a floor having at least one upstanding sidewall for supporting the scaffold.

22-42. (canceled)
